(19) United States
(12) Patent Application Publication
(10) Pub. No.: US 2023/0382972 A9
(43) Pub. Date: Nov. 30, 2023

(54) CD80 EXTRACELLULAR DOMAIN FC FUSION PROTEIN REGIMENS

(71) Applicant: FIVE PRIME THERAPEUTICS, INC., Thousand Oaks, CA (US)

(72) Inventors: Hong XIANG, Thousand Oaks, CA (US); Siddhartha MITRA, Thousand Oaks, CA (US)

(21) Appl. No.: 17/773,800

(22) PCT Filed: Nov. 4, 2020
(86) PCT No.: PCT/US2020/058972

§ 371 Date: May 2, 2022

Related U.S. Application Data
(60) Provisional application No. 62/930,334, filed on Nov. 4, 2019.

(51) Int. Cl. C07K 14/705; A61P 35/00

(52) U.S. Cl. CPC C07K 14/70532; A61P 35/00; A61K 2039/505

(57) ABSTRACT

The present disclosure provides methods of administering fusion proteins comprising the extracellular domain of human cluster of differentiation 80 (CD80) and the fragment crystallizable (Fc) domain of human immunoglobulin G1 (IgG1) to a subject in need thereof, for example, a cancer patient.

### CROSS REFERENCE TO RELATED APPLICATIONS

The application claims the benefit of U.S. Provisional Application No. 62/930,334, filed Nov. 4, 2019, which is hereby incorporated by reference in its entirety.

### FIELD

This application relates to dosing regimens for fusion proteins comprising an CD80 (B7-1) extracellular domain (ECD) and an immunoglobulin fragment crystallizable (Fc) domain for the treatment of cancer.

### BACKGROUND

T-cell regulation involves the integration of multiple signaling pathways: signaling via the T-cell receptor (TCR) complex and through co-signaling receptors, both co-stimulatory and co-inhibitory. CD80 (cluster of differentiation 80, also known as B7, B7.1, B7-1) is a well-characterized co-signaling ligand. It is expressed on professional antigen-presenting cells (APCs) such as dendritic cells and activated macrophages. Following TCR recognition of cognate peptide-major histocompatibility complex (MHC), CD80 acts as a co-stimulatory ligand via interactions with its receptor, cluster of differentiation 28 (CD28), expressed on T-cells. In addition to signaling via CD28, CD80 also interacts with co-inhibitory molecules cytotoxic T-lymphocyte-associated antigen-4 (CTLA-4) and programmed death-ligand 1 (PD-L1). CD80 interactions with CTLA-4 are central for dampening the T-cell response once activated T-cell responses are no longer needed, while the biological significance of the CD80 interaction with PD-L1 is not as well understood. Together, the co-stimulatory and co-inhibitory ligands ensure both tolerance to self-antigens and the ability to mount an appropriate immune response to non-self antigens.

Although the immune system is often initially able to mount an effective immune response against tumor cells via TCR-dependent and -independent mechanisms, some tumors can evade the immune response. Mechanisms by which this occurs include the upregulation of pathways that enforce peripheral tolerance to self-antigens (including CTLA-4 and PD-L1). Recent immuno-oncology approaches have focused on reprogramming the immune system to mount an effective immune response against tumors that have evaded the initial immune response. These approaches include the use of “checkpoint inhibitors.” For example, blocking antibodies against both the programmed cell death protein (PD-1)/PD-L1 and CTLA-4 axes have been effective in anti-tumor immunity, including improved progression free survival (PFS) and overall survival (OS) in some patients. However, responses have only been observed in select tumor types, within which only a fraction of patients respond to checkpoint inhibitors. Although some patients do achieve long term disease control with the use of blocking antibodies against the PD-1/PD-L1 and CTLA-4 axes, the majority of patients either do not respond or respond then subsequently relapse. Therefore, a need exists for additional immuno-oncology approaches, and the CD80 signaling axis may provide additional opportunities for intervention.

### SUMMARY

Methods of administering a fusion protein comprising the extracellular domain (ECD) of human cluster of differentiation 80 (CD80) and the fragment crystallizable (Fc) domain of human immunoglobulin G 1 (IgG1) using a therapeutically effective and safe dose regimen are provided herein. As described herein, these methods take into account multiple factors that make dosing of such fusion proteins particularly challenging, including, for example: the complex mechanism of action of CD80, which involves the interaction of CD80 with three different receptors having different affinities (wherein the biological significance of one of these interactions remains unclear); and the potential for toxic effects uniquely associated with the mechanism of action of CD80 and its receptors, including cytokine release syndrome (CRS) and other undesired effects.

In certain aspects, a method of treating a solid tumor in a human patient comprises administering to the patient about 140 mg to about 1,260 mg of a fusion protein comprising the ECD of human CD80 and the Fc domain of human IgG1. In certain aspects, about 280 mg to about 1,260 mg of the fusion protein is administered. In certain aspects, a method of treating a solid tumor in a human patient comprises administering to the patient about 140 mg to about 700 mg of a fusion protein comprising the ECD of human CD80 and the Fe domain of human IgG1. In certain aspects, about 280 mg to about 700 mg of the fusion protein is administered. In certain aspects, a method of treating a solid tumor in a human patient comprises administering to the patient about 140 mg to about 210 mg of a fusion protein comprising the ECD of human CD80 and the Fe domain of human IgG1. In certain aspects, a method of treating a solid tumor in a human patient comprises administering to the patient about 210 mg to about 280 mg of a fusion protein comprising the ECD of human CD80 and the Fc domain of human IgG1. In certain aspects, a method of treating a solid tumor in a human patient comprises administering to the patient about 280 mg to about 420 mg of a fusion protein comprising the ECD of human CD80 and the Fc domain of human IgG1. In certain aspects, a method of treating a solid tumor in a human patient comprises administering to the patient about 420 mg to about 560 mg of a fusion protein comprising the ECD of human CD80 and the Fc domain of human IgG1. In certain aspects, a method of treating a solid tumor in a human patient comprises administering to the patient about 560 mg to about 630 mg of a fusion protein comprising the ECD of human CD80 and the Fc domain of human IgG1. In certain aspects, a method of treating a solid tumor in a human patient comprises administering to the patient about 630 mg to about 700 mg of a fusion protein comprising the ECD of human CD80 and the Fc domain of human IgG1. In certain aspects, a method of treating a solid tumor in a human patient comprises administering to the patient about 700 mg to about 840 mg of a fusion protein comprising the ECD of human CD80 and the Fc domain of human IgG1. In certain aspects, a method of treating a solid tumor in a human patient comprises administering to the patient about 840 mg to about 1,260 of a fusion protein comprising the ECD of human CD80 and the Fc domain of human IgG1.

In certain aspects, about 1,260 mg of the fusion protein is administered. In certain aspects, about 840 mg of the fusion protein is administered. In certain aspects, about 700 mg of the fusion protein is administered. In certain aspects, about 630 mg of the fusion protein is administered. In certain aspects, about 560 mg of the fusion protein is administered. In certain aspects, about 420 mg of the fusion protein is administered. In certain aspects, about 280 mg of the fusion protein is administered. In certain aspects, about 210 mg of the fusion protein is administered. In certain aspects, about 140 mg of the fusion protein is administered.

In certain aspects, the fusion protein is administered once every three weeks.

In certain aspects, the fusion protein is administered intravenously.

In certain aspects, the ECD of human CD80 comprises the amino acid sequence set forth in SEQ ID NO:1. In certain aspects, the Fc domain of human IgG1 comprises the amino acid sequence set forth in SEQ ID NO:3. In certain aspects, the Fc domain of human IgG1 is linked to the carboxy terminus of the ECD of human CD80. In certain aspects, the fusion protein comprises the amino acid sequence set forth in SEQ ID NO:5.

In certain aspects, the fusion protein comprises at least 20 molecules of sialic acid (SA). In certain aspects, the fusion protein comprises at least 15 molecules of SA. In certain aspects, the fusion protein comprises 15-60 molecules of SA. In certain aspects, the fusion protein comprises 15-40 molecules of SA. In certain aspects, the fusion protein comprises 15-30 molecules of SA. In certain aspects, the fusion protein comprises 15-26 molecules of SA. In certain aspects, the fusion protein comprises 20-30 molecules of SA. In certain aspects, the fusion protein comprises 20-26 molecules of SA.

In certain aspects, the fusion protein is administered in a pharmaceutical composition that further comprises a pharmaceutically acceptable excipient. In certain aspects, the pharmaceutical composition comprises at least 20 moles of SA per mole of fusion protein. In certain aspects, the pharmaceutical composition comprises at least 15 moles of SA per mole of fusion protein. In certain aspects, the pharmaceutical composition comprises 15-60 moles of SA per mole of fusion protein. In certain aspects, the pharmaceutical composition comprises 15-40 moles of SA per mole of fusion protein. In certain aspects, the pharmaceutical composition comprises 15-30 moles of SA per mole of fusion protein. In certain aspects, the pharmaceutical composition comprises 15-26 moles of SA per mole of fusion protein. In certain aspects, the pharmaceutical composition comprises 20-30 moles of SA per mole of fusion protein. In certain aspects, the pharmaceutical composition comprises 20-26 moles of SA per mole of fusion protein.

In certain aspects, the solid tumor is an advanced solid tumor. In certain aspects, the solid tumor is not a primary central nervous system tumor. In certain aspects, the solid tumor is a colorectal cancer, breast cancer, gastric cancer, non-small cell lung cancer, small cell lung cancer, melanoma, squamous cell carcinoma of the head and neck, ovarian cancer, pancreatic cancer, renal cell carcinoma, hepatocellular carcinoma, bladder cancer, or endometrial cancer. In certain aspects, the solid tumor is a renal cell carcinoma. In certain aspects, the solid tumor is a melanoma. In certain aspects, the solid tumor is a sarcoma.

In certain aspects, the patient has not received prior therapy with a PD-1/PD-L1 antagonist. In certain aspects, the patient has received prior therapy with at least one PD-1/PD-L1 antagonist selected from a PD-L1 antagonist and a PD-1 antagonist. In certain aspects, the PD-1/PD-L1 antagonist is nivolumab, pembrolizumab, atezolizumab, durvalumab, or avelumab. In certain aspects, the at least one PD-1/PD-1 antagonist was administered in an advanced or metastatic setting.

In certain aspects, the patient has received prior therapy with at least one anti-angiogenic agent. In certain aspects, the anti-angiogenic agent is sunitinib, sorafenib, pazopanib, axitinib, tivozanib, ramucirumab, or bevacizumab. In certain aspects, the at least one anti-angiogenic agent was administered in an advanced or metastatic setting.

In certain aspects, the patient (e.g., a patient with melanoma) has a BRAF mutation. In certain aspects, the patient has received prior therapy with at least one BRAF inhibitor. In certain aspects, the BRAF inhibitor is vemurafenib or dabrafenib. In certain aspects, the BRAF inhibitor was administered in an advanced or metastatic setting.

In certain aspects, the solid tumor is recurrent or progressive after a therapy selected from surgery, chemotherapy, radiation therapy, and a combination thereof.

### BRIEF DESCRIPTION OF THE DRAWINGS

FIGS. 1A-D show release of cytokines IFN-γ and TNF-α from T-cells on 96-well tissue culture plates exposed to protein A beads coated with 0.01, 0.1, or 1 μg/well of a CD80 ECD IgG1 Fc domain fusion molecule (CD80-Fc). FIGS. 1A and 1C show that bead-immobilized CD80-Fc alone did not cause significant T-cell activation, as measured by soluble cytokine production. FIGS. 1B and 1D show that when a small amount of OKT3-scFv (too low to cause T-cell stimulation on its own) was immobilized along with the CD80-Fc, cytokine release was observed. (See Example 1.)

FIG. 2 shows tumor growth of murine CT26 tumors following treatment with a saline control or either 0.3 or 0.6 mg/kg doses of three different lots of a CD80 ECD-Fc fusion molecule having three different sialic acid (SA) contents. Lot A has 5 mol SA/mol protein, lot D has 15 mol SA/mol protein and lot E has 20 mol SA/mol protein. Treatment with CD80 ECD-Fc lot E dosed at 0.3 or 0.6 mg/kg resulted in a 93% and 98% inhibition of tumor growth compared to the control (P<0.001). Treatment with CD80 ECD-Fc lot D dosed at 0.3 or 0.6 mg/kg resulted in a 93% and 95% inhibition of tumor growth compared to the control (P<0.001). By comparison, treatment with CD80 ECD-Fc lot A at 0.3 mg/kg did not inhibit tumor growth compared to the control and when dosed at 0.6 mg/kg it only induced 70% inhibition (P<0.001) of tumor growth. (See Example 2.)

FIG. 3 shows tumor growth of CT26 tumors treated with mouse IgG2b at 10 mg/kg; murine CD80 ECD-Fc SA 20 mol/mol at 0.3 mg/kg; anti-CTLA4 antibody clone 9D9 at 10 mg/kg; and anti-CTLA4 antibody clone 9D9 at 1.5 mg/kg. Arrows indicate when mice were dosed. The asterisk symbol (*) denotes statistically significant differences between murine CD80 ECD-Fc SA 20 mol/mol at 0.3 mg/kg and the other treatments. (See Example 3.)

FIG. 4 shows tumor growth of MC38 tumors treated with mouse IgG2b at 10 mg/kg; murine CD80 ECD-Fc SA 20 mol/mol at 3 mg/kg; anti-CTLA4 antibody clone 9D9 at 10 mg/kg; and anti-CTLA4 antibody clone 9D9 at 1.5 mg/kg. Arrows indicate when mice were dosed. The asterisk symbol (*) denotes statistically significant differences between murine CD80 ECD-Fc SA 20 mol/mol at 3 mg/kg and the other treatments. (See Example 3.)

FIG. 5 shows tumor growth of B16 tumors treated with mouse IgG2b at 10 mg/kg; murine CD80 ECD-Fc SA 20 mol/mol at 3 mg/kg; anti-CTLA4 antibody clone 9D9 at 10 mg/kg; and anti-CTLA4 antibody clone 9D9 at 1.5 mg/kg. Arrows indicate when mice were dosed. The asterisk symbol (*) denotes statistically significant differences between murine CD80 ECD-Fc SA 20 mol/mol at 3 mg/kg and the other treatments. (See Example 3.)

FIG. 6 shows the Phase 1A and 1B study schema. DLT=dose-limiting toxicity; RCC=renal cell carcinoma; RD=recommended dose. (See Examples 8 and 9.)

FIG. 7 shows normalized expression of granzyme B (Gzmb) and interferon gamma (Ifng) in the tumor cells and in the blood of BALB/c mice inoculated with CT26 colorectal carcinoma cells and in the blood of naïve BALB/c mice. The CT26 tumor-bearing mice and the naïve mice received either mIgG2a (control) or a dose of murine CD80 ECD-Fc. The asterisk symbol (*p<0.05) or (**p<0.01) denotes statistically significant differences between murine CD80 ECD-Fc compared to control treatment. (See Example 10).

FIGS. 8A and B show hCD80ECD:hIgG1Fc-induced stimulator-dependent allogeneic T cell cytokine secretion. HCD80ECD: hIgG1Fc-enhanced allogenic induction of IL-2 (8A) and IFNγ (8B) in culture supernatants. Whole blood was added to two amounts of pooled, irradiated PBMC and cultured for 5 days following the addition of multiple doses of Fc-Hinge control or hCD80ECD:hIgG1Fc. All data are mean±SD of the mean of 6 technical replicates from 6 individual donors. Statistical analyses are 1-way ANOVA with Kruskal-Wallis post-test where * p<0.05. (See Example 11).

FIGS. 9A and 9B show hCD80ECD:hIgG1Fc-induced stimulator-dependent T cell costimulation. (9A) Increased proliferation of CD4 and CD8 T cells stimulated with hCD80ECD:hIgG1Fc as determined by EdU incorporation. (9B) Upregulation of CD25 following hCD80ECD:hIgG1Fc stimulation. Whole blood was added to two amounts of pooled, irradiated PBMC and cultured for 5 days following the addition of multiple doses of Fc-Hinge control or hCD80ECD:hIgG1Fc. Following the removal of the supernatant on day 5 post culture, additional media containing EdU was added to the culture. After 24 hours the cells were collected, stained with surface antibodies, fixed, permeabilized, and stained for Click-iT EdU kit reaction to label EdU. All data are mean±SD of the mean of 6 technical replicates from 6 individual donors. Statistical analyses are 1-way ANOVA with Kruskal-Wallis post-test where * p<0.05, ** p<0.01. (See Example 11.)

FIGS. 10A and 10B shows the impact of murine CD80 ECD-Fc on the growth of CT26 tumors. The average tumor growth (FIG. 10A) and individual tumor volumes of all groups on day 21 (FIG. 10B) are shown. Immunocompetent BALB/c mice were inoculated with 1×10⁶ CT26 tumor cells. Treatment with murine CD80 ECD-Fc was initiated on day 10; three doses were administered on days 10, 13, and 17. Murine CD80 ECD-Fc significantly inhibited tumor growth (**** indicates p<0.0001 for 0.3 mg/kg; ** indicates p<0.01 for 1 mg/kg, and *** p<0.001 for 3 mg/kg). Statistical significance was determined by 1-way ANOVA. Abbreviations: SD=standard deviation. (See Example 12.)

FIG. 11 shows the serum concentration versus time profiles of hCD80ECD:hIgG1Fc in patients who received a single intravenous infusion administration of hCD80ECD:hIgG1Fc in the dose range of 0.07 mg to 42 mg.

FIG. 12 shows the plasma concentration of CD80 ECD-Fc fusion protein with different amounts of sialic acid in Balb/c mice over time after administration of a 5 mg/kg dose. (See Example 14.)

FIG. 13 shows the serum concentration versus time profiles of hCD80ECD:hIgG1Fc in patients who received a single intravenous infusion administration of hCD80ECD:hIgG1Fc in the dose range of 0.07 mg to 560 mg. (See Example 15.)

FIG. 14 shows adverse effects in human patients who received hCD80ECD:hIgG1Fc. (See Example 15.)

### DESCRIPTION OF PARTICULAR EMBODIMENTS

### 1. Definitions

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular.

Unless specifically stated or obvious from context, as used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The terms “polypeptide,” “peptide,” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present invention, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

A “fusion molecule” as used herein refers to a molecule composed of two or more different molecules that do not occur together in nature being covalently or noncovalently joined to form a new molecule. For example, fusion molecules may be comprised of a polypeptide and a polymer such as PEG, or of two different polypeptides. A “fusion protein” refers to a fusion molecule composed of two or more polypeptides that do not occur in a single molecule in nature.

A “CD80 extracellular domain” or “CD80 ECD” refers to an extracellular domain polypeptide of CD80, including natural and engineered variants thereof. A CD80 ECD can, for example, comprise, consist essentially of, or consist of the amino acid sequence set forth in SEQ ID NO:1 or 2. A “CD80 ECD fusion molecule” refers to a molecule comprising a CD80 ECD and a fusion partner. The fusion partner may be covalently attached, for example, to the N- or C-terminal of the CD80 ECD or at an internal location. A “CD80 ECD fusion protein” is a CD80 ECD fusion molecule comprising a CD80 ECD and another polypeptide that is not naturally associated with the CD80 ECD, such as an Fc domain. A CD80 ECD fusion protein can, for example, comprise, consist essentially of, or consist of the amino acid sequence set forth in SEQ ID NO: 4 or 5.

The term “isolated” as used herein refers to a molecule that has been separated from at least some of the components with which it is typically found in nature. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, e.g., in the case of an RNA polynucleotide. Thus, a DNA polynucleotide that is contained in a vector inside a host cell may be referred to as “isolated” so long as that polynucleotide is not found in that vector in nature.

The terms “subject” and “patient” are used interchangeably herein to refer to a human. In some embodiments, methods of treating other mammals, including, but not limited to, rodents, simians, felines, canines, equines, bovines, porcines, ovines, caprines, mammalian laboratory animals, mammalian farm animals, mammalian sport animals, and mammalian pets, are also provided.

The term “cancer” is used herein to refer to a group of cells that exhibit abnormally high levels of proliferation and growth. A cancer can be a solid tumor, for example, a colorectal cancer, breast cancer, gastric cancer, non-small cell lung cancer, small cell lung cancer, melanoma, squamous cell carcinoma of the head and neck, ovarian cancer, pancreatic cancer, renal cell carcinoma, hepatocellular carcinoma, bladder cancer, endometrial cancer, or sarcoma.

Terms such as “treating,” “treatment,” and “to treat,” refer to therapeutic measures that cure, slow down, lessen symptoms of, and/or halt progression of a pathologic condition or disorder. Thus, those in need of treatment include those already diagnosed with or suspected of having the disorder. In certain embodiments, a subject is successfully “treated” for cancer according to the methods of the present invention if the patient shows one or more of the following: a reduction in the number of or complete absence of cancer cells; a reduction in the tumor size; inhibition of or an absence of cancer cell infiltration into peripheral organs including, for example, the spread of cancer into soft tissue and bone; inhibition or an absence of tumor metastasis; inhibition or an absence of tumor growth; relief of one or more symptoms associated with the specific cancer; reduced morbidity and mortality; improvement in quality of life; reduction in tumorigenicity, tumorigenic frequency, or tumorigenic capacity, of a tumor; reduction in the number or frequency of cancer stem cells in a tumor; differentiation of tumorigenic cells to a non-tumorigenic state; increased progression-free survival (PFS), disease-free survival (DFS), overall survival (OS), complete response (CR), partial response (PR), stable disease (SD), a decrease in progressive disease (PD), a reduced time to progression (TTP), or any combination thereof.

The terms “administer,” “administering,” “administration,” and the like, as used herein, refer to methods that may be used to enable delivery of a drug, e.g., a CD80 ECD fusion protein to the desired site of biological action (e.g., intravenous administration). Administration techniques that can be employed with the agents and methods described herein are found in e.g., Goodman and Gilman, The Pharmacological Basis of Therapeutics, current edition, Pergamon; and Remington's, Pharmaceutical Sciences, current edition, Mack Publishing Co., Easton, Pa.

The term “therapeutically effective amount” refers to an amount of a drug, e.g., a CD80 ECD fusion protein, effective to treat a disease or disorder in a subject. In the case of cancer, the therapeutically effective amount of the drug can reduce the number of cancer cells; reduce the tumor size or burden; inhibit, to some extent, cancer cell infiltration into peripheral organs; inhibit, to some extent, tumor metastasis; inhibit, to some extent, tumor growth; relieve, to some extent, one or more of the symptoms associated with the cancer; and/or result in a favorable response such as increased progression-free survival (PFS), disease-free survival (DFS), overall survival (OS), complete response (CR), partial response (PR), or, in some cases, stable disease (SD), a decrease in progressive disease (PD), a reduced time to progression (TTP), or any combination thereof.

The terms “resistant” or “nonresponsive” when used in the context of treatment with a therapeutic agent, means that the subject shows decreased response or lack of response to a standard dose of the therapeutic agent, relative to the subject's response to the standard dose of the therapeutic agent in the past, or relative to the expected response of a similar subject with a similar disorder to the standard dose of the therapeutic agent. Thus, in some embodiments, a subject may be resistant to a therapeutic agent although the subject has not previously been given the therapeutic agent, or the subject may develop resistance to the therapeutic agent after having responded to the agent on one or more previous occasions.

A “refractory” cancer is one that progresses even though an anti-tumor treatment, such as a chemotherapy, is administered to the cancer patient.

A “recurrent” cancer is one that has regrown, either at the initial site or at a distant site, after a response to initial therapy.

The terms “programmed cell death protein 1” and “PD-1” refer to an immunoinhibitory receptor belonging to the CD28 family. PD-1 is expressed predominantly on previously activated T-cells in vivo, and binds to two ligands, PD-L1 and PD-L2. The term “PD-1” as used herein includes human PD-1 (hPD-1), naturally occurring variants and isoforms of hPD-1, and species homologs of hPD-1. A mature hPD-1 sequence is provided as SEQ ID NO:6.

The terms “programmed cell death 1 ligand 1” and “PD-L1” refer to one of two cell surface glycoprotein ligands for PD-1 (the other being PD-L2) that down regulate T-cell activation and cytokine secretion upon binding to PD-1. The term “PD-L1” as used herein includes human PD-L1 (hPD-L1), naturally occurring variants and isoforms of hPD-1, and species homologs of hPD-L1. A mature hPD-L1 sequence is provided as SEQ ID NO:7.

The term “PD-1/PD-L1 antagonist” refers to a moiety that disrupts the PD-1/PD-L1 signaling pathway. In some embodiments, the antagonist inhibits the PD-1/PD-L1 signaling pathway by binding to PD-1 and/or PD-L1. In some embodiments, the PD-1/PD-L1 antagonist also binds to PD-L2. In some embodiments, a PD-1/PD-L1 antagonist blocks binding of PD-1 to PD-L1 and optionally PD-L2. Nonlimiting exemplary PD-1/PD-L1 antagonists include PD-1 antagonists, such as antibodies that bind to PD-1 (e.g., nivolumab and pembrolizumab); PD-L1 antagonists, such as antibodies that bind to PD-L1 (e.g., atezolizumab, durvalumab and avelumab); fusion proteins, such as AMP-224; and peptides, such as AUR-012.

An “anti-angiogenic agent” or “angiogenesis inhibitor” refers to an agent such as a small molecular weight substance, a polynucleotide (including, e.g., an inhibitory RNA (RNAi or siRNA)), a polypeptide, an isolated protein, a recombinant protein, an antibody, or conjugates or fusion proteins thereof, that inhibits angiogenesis, vasculogenesis, or undesirable vascular permeability, either directly or indirectly. It should be understood that an anti-angiogenic agent includes those agents that bind and block the angiogenic activity of the angiogenic factor or its receptor. For example, an anti-angiogenic agent is an antibody to or other antagonist of an angiogenic agent, e.g., antibodies to VEGF-A (e.g., bevacizumab (Avastin®)) or to the VEGF-A receptor (e.g., KDR receptor or Flt-1 receptor), anti-PDGFR inhibitors such as Gleevec® (imatinib mesylate), small molecules that block VEGF receptor signaling (e.g., PTK787/ZK2284, SU6668, Sutent®/SU11248 (sunitinib malate), AMG706, or those described in, e.g., international patent application WO 2004/113304). Anti-angiogensis agents also include native angiogenesis inhibitors, e.g., angiostatin, endostatin, etc. See, e.g., Klagsbrun and D′Amore (1991) Annu. Rev. Physiol. 53:217-39; Streit and Detmar (2003) Oncogene 22:3172-3179 (e.g., Table 3 listing anti-angiogenic therapy in malignant melanoma); Ferrara & Alitalo (1999) Nature Medicine 5(12):1359-1364; Tonini et al. (2003) Oncogene 22:6549-6556 (e.g., Table 2 listing known anti-angiogenic factors); Sato (2003) Int. J. Clin. Oncol. 8:200-206 (e.g., Table 1 listing anti-angiogenic agents used in clinical trials), and Jayson (2016) Lancet 338(10043):518-529.

The term “pharmaceutical composition” refers to a preparation which is in such form as to permit the biological activity of the active ingredient to be effective, and which contains no additional components which are unacceptably toxic to a subject to which the formulation would be administered. The formulation can be sterile. A pharmaceutical composition may contain a “pharmaceutical carrier,” which refers to carrier that is non-toxic to recipients at the dosages and concentrations employed and is compatible with other ingredients of the formulation. The pharmaceutically acceptable carrier is appropriate for the formulation employed. For example, if the therapeutic agent is to be administered intravenously, the carrier ideally is not irritable to the skin and does not cause injection site reaction.

As used herein, the terms “about” and “approximately,” when used to modify a numeric value or numeric range, indicate that deviations of 5% to 10% above and 5% to 10% below the value or range remain within the intended meaning of the recited value or range.

Any compositions or methods provided herein can be combined with one or more of any of the other compositions and methods provided herein.

### 2. CD80 Extracellular Domain Fc Fusion Proteins

Provided herein are methods of administering CD80 ECD fusion proteins comprising a CD80 ECD and an Fc domain (a “CD80 ECD-Fc fusion protein”).

The CD80 ECD can, for example, be a human CD80 ECD. In certain aspects, the human CD80 ECD comprises, consists essentially of, or consists of the amino acid sequence set forth in SEQ ID NO:1.

The Fc domain can be the Fc domain of an IgG. The Fc domain can be the Fc domain of a human immunoglobulin. In certain aspects, the Fc domain is a human IgG Fc domain. In certain aspects, the Fc domain is a human IgG1 Fc domain. In certain aspects, the human IgG1 Fe domain comprises, consists essentially of, or consists of the amino acid sequence set forth in SEQ ID NO:4.

The CD80 ECD and the Fc domain can be directly linked such that the N-terminal amino acid of the Fc domain immediately follows the C-terminal amino acid of the CD80 ECD. In certain aspects, the CD80 ECD and the Fc domain are translated as a single polypeptide from a coding sequence that encodes both the CD80 ECD and the Fc domain. In certain aspects, the Fc domain is directly fused to the carboxy-terminus of the CD80 ECD polypeptide. In certain aspects, the CD80 ECD-Fc fusion protein comprises a human CD80 ECD and a human IgG1 Fc domain. In certain aspects, the CD80 ECD-Fc fusion protein comprises, consists essentially of, or consists of the amino acid sequence set forth in SEQ ID NO:5.

CD80 ECD-Fc fusion proteins can, depending on how they are produced, have different levels of particular glycosylation modifications. For example, a CD80 ECD-Fc fusion protein can have different amounts of sialic acid (SA) residues.

In certain aspects, a CD80 ECD-Fc fusion protein (e.g., comprising SEQ ID NO:5) comprises 10 to 60 molecules of SA. In certain aspects, a CD80 ECD-Fc fusion protein (e.g., comprising SEQ ID NO:5) comprises 15 to 60 molecules of SA. In certain aspects, a CD80 ECD-Fc fusion protein (e.g., comprising SEQ ID NO:5) comprises 10 to 40 molecules of SA. In certain aspects, a CD80 ECD-Fc fusion protein (e.g., comprising SEQ ID NO:5) comprises 15 to 30 molecules of SA. In certain aspects, a CD80 ECD-Fc fusion protein (e.g., comprising SEQ ID NO:5) comprises 15 to 25 molecules of SA. In certain aspects, a CD80 ECD-Fc fusion protein (e.g., comprising SEQ ID NO:5) comprises 15 to 26 molecules of SA. In certain aspects, a CD80 ECD-Fc fusion protein (e.g., comprising SEQ ID NO:5) comprises 20 to 40 molecules of SA. In certain aspects, a CD80 ECD-Fc fusion protein (e.g., comprising SEQ ID NO:5) comprises 20 to 30 molecules of SA. In certain aspects, a CD80 ECD-Fc fusion protein (e.g., comprising SEQ ID NO:5) comprises 20 to 26 molecules of SA. In certain aspects, a CD80 ECD-Fc fusion protein (e.g., comprising SEQ ID NO:5) comprises 30 to 40 molecules of SA. In certain aspects, a CD80 ECD-Fc fusion protein (e.g., comprising SEQ ID NO:5) comprises 10, 15, 20, 25, 26, 30, 35, or 40 molecules of SA. In certain aspects, a CD80 ECD-Fc fusion protein (e.g., comprising SEQ ID NO:5) comprises 10, 15, 20, 25, 30, 35, or 40 molecules of SA. In certain aspects, a CD80 ECD-Fc fusion protein (e.g., comprising SEQ ID NO:5) comprises at least 15 molecules of SA. In certain aspects, a CD80 ECD-Fc fusion protein (e.g., comprising SEQ ID NO:5) comprises at least 20 molecules of SA. In certain aspects, a CD80 ECD-Fc fusion protein (e.g., comprising SEQ ID NO:5) comprises at least 25 molecules of SA. In certain aspects, a CD80 ECD-Fc fusion protein (e.g., comprising SEQ ID NO:5) comprises at least 26 molecules of SA. In certain aspects, a CD80 ECD-Fc fusion protein (e.g., comprising SEQ ID NO:5) comprises at least 30 molecules of SA. In certain aspects, a CD80 ECD-Fc fusion protein (e.g., comprising SEQ ID NO:5) comprises at least 35 molecules of SA. In certain aspects, a CD80 ECD-Fc fusion protein (e.g., comprising SEQ ID NO:5) comprises at least 40 molecules of SA.

CD80 ECD-Fc fusion proteins can directly engage CD28 through the CD80 ECD. This can lead to direct activation of naïve and memory T cells. Accordingly, in certain aspects a CD80 ECD-Fc fusion protein (e.g., comprising SEQ ID NO:5) is capable of activating naïve and memory T cells. In certain aspects a CD80 ECD-Fc fusion protein (e.g., comprising SEQ ID NO:5) is capable of directly activating naïve and memory T cells.

CD80 ECD-Fc fusion proteins can also bind to CTLA-4 through the CD80 ECD. This can cause de-repressing T-cell activation by enabling the interaction of endogenous CD20 with CD28 at the immune synapse.

In certain aspects a CD80 ECD-Fc fusion protein (e.g., comprising SEQ ID NO:5) is capable of binding to CD28. In certain aspects a CD80 ECD-Fc fusion protein (e.g., comprising SEQ ID NO:5) is capable of binding to CTLA-4. In certain aspects a CD80 ECD-Fc fusion protein (e.g., comprising SEQ ID NO:5) is capable of binding to CD28 and CTLA-4.

In certain aspects a CD80 ECD-Fc fusion protein (e.g., comprising SEQ ID NO:5) is not a CD28 superagonist. In certain aspects, a CD80 ECD-Fc fusion protein (e.g., comprising SEQ ID NO:5) is at least 1000-fold less potent at inducing cytokine release compared to TGN1412.

### 3. Pharmaceutical Compositions Comprising CD80 Extracellular Domain Fc Fusion Proteins

Provided herein are methods of administering pharmaceutical compositions comprising CD80 ECD-Fc fusion proteins, e.g. having the desired degree of purity in a physiologically acceptable carrier, excipient, or stabilizer (Remington's Pharmaceutical Sciences (1990) Mack Publishing Co., Easton, Pa.). Acceptable carriers, excipients, or stabilizers are nontoxic to recipients at the dosages and concentrations employed. (See, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). The compositions to be used for in vivo administration can be sterile. This is readily accomplished by filtration through, e.g., sterile filtration membranes.

In certain aspects, a pharmaceutical composition comprising a CD80 ECD-Fc fusion protein (e.g. comprising SEQ ID NO:5) is formulated for intravenous administration.

In certain aspects, a pharmaceutical composition comprises 1,260 mg of a CD80 ECD-Fc fusion protein (e.g. comprising SEQ ID NO:5). In certain aspects, a pharmaceutical composition comprises 840 mg of a CD80 ECD-Fc fusion protein (e.g. comprising SEQ ID NO:5). In certain aspects, a pharmaceutical composition comprises 700 mg of a CD80 ECD-Fc fusion protein (e.g. comprising SEQ ID NO:5). In certain aspects, a pharmaceutical composition comprises 630 mg of a CD80 ECD-Fc fusion protein (e.g. comprising SEQ ID NO:5). In certain aspects, a pharmaceutical composition comprises 560 mg of a CD80 ECD-Fc fusion protein (e.g. comprising SEQ ID NO:5). In certain aspects, a pharmaceutical composition comprises 420 mg of a CD80 ECD-Fc fusion protein (e.g. comprising SEQ ID NO:5). In certain aspects, a pharmaceutical composition comprises 280 mg of a CD80 ECD-Fc fusion protein (e.g. comprising SEQ ID NO:5). In certain aspects, a pharmaceutical composition comprises 210 mg of a CD80 ECD-Fc fusion protein (e.g. comprising SEQ ID NO:5). In certain aspects, a pharmaceutical composition comprises 140 mg of a CD80 ECD-Fc fusion protein (e.g. comprising SEQ ID NO:5).

In certain aspects, a pharmaceutical composition comprises 140 to 1,260 mg of a CD80 ECD-Fc fusion protein (e.g. comprising SEQ ID NO:5). In certain aspects, a pharmaceutical composition comprises 280 to 1,260 mg of a CD80 ECD-Fc fusion protein (e.g. comprising SEQ ID NO:5). In certain aspects, a pharmaceutical composition comprises 140 to 700 mg of a CD80 ECD-Fc fusion protein (e.g. comprising SEQ ID NO:5). In certain aspects, a pharmaceutical composition comprises 280 to 700 mg of a CD80 ECD-Fc fusion protein (e.g. comprising SEQ ID NO:5). In certain aspects, a pharmaceutical composition comprises 140 to 210 mg of a CD80 ECD-Fc fusion protein (e.g. comprising SEQ ID NO:5). In certain aspects, a pharmaceutical composition comprises 210 to 280 mg of a CD80 ECD-Fc fusion protein (e.g. comprising SEQ ID NO:5). In certain aspects, a pharmaceutical composition comprises 280 to 420 mg of a CD80 ECD-Fc fusion protein (e.g. comprising SEQ ID NO:5). In certain aspects, a pharmaceutical composition comprises 420 to 560 mg of a CD80 ECD-Fc fusion protein (e.g. comprising SEQ ID NO:5). In certain aspects, a pharmaceutical composition comprises 560 to 630 mg of a CD80 ECD-Fc fusion protein (e.g. comprising SEQ ID NO:5). In certain aspects, a pharmaceutical composition comprises 630 to 700 mg of a CD80 ECD-Fc fusion protein (e.g. comprising SEQ ID NO:5). In certain aspects, a pharmaceutical composition comprises 700 mg to 840 mg of a CD80 ECD-Fc fusion protein (e.g. comprising SEQ ID NO:5). In certain aspects, a pharmaceutical composition comprises 840 mg to 1,260 mg of a CD80 ECD-Fc fusion protein (e.g. comprising SEQ ID NO:5).

In certain aspects a pharmaceutical composition comprises CD80 ECD-Fc fusion proteins (e.g. comprising SEQ ID NO:5) comprising 10 to 60 moles of SA per mole CD80 ECD-Fc fusion protein. In certain aspects a pharmaceutical composition comprises CD80 ECD-Fc fusion proteins (e.g. comprising SEQ ID NO:5) comprising 15 to 60 moles of SA per mole CD80 ECD-Fc fusion protein. In certain aspects a pharmaceutical composition comprises CD80 ECD-Fc fusion proteins (e.g. comprising SEQ ID NO:5) comprising 10 to 40 moles of SA per mole CD80 ECD-Fc fusion protein. In certain aspects a pharmaceutical composition comprises CD80 ECD-Fc fusion proteins (e.g. comprising SEQ ID NO:5) comprising 15 to 30 moles of SA per mole CD80 ECD-Fc fusion protein. In certain aspects a pharmaceutical composition comprises CD80 ECD-Fc fusion proteins (e.g. comprising SEQ ID NO:5) comprising 15 to 25 moles of SA per mole CD80 ECD-Fc fusion protein. In certain aspects a pharmaceutical composition comprises CD80 ECD-Fc fusion proteins (e.g. comprising SEQ ID NO:5) comprising 15 to 26 moles of SA per mole CD80 ECD-Fc fusion protein. In certain aspects a pharmaceutical composition comprises CD80 ECD-Fc fusion proteins (e.g. comprising SEQ ID NO:5) comprising 20 to 40 moles of SA per mole CD80 ECD-Fc fusion protein. In certain aspects a pharmaceutical composition comprises CD80 ECD-Fc fusion proteins (e.g. comprising SEQ ID NO:5) comprising 20 to 30 moles of SA per mole CD80 ECD-Fc fusion protein. In certain aspects a pharmaceutical composition comprises CD80 ECD-Fc fusion proteins (e.g. comprising SEQ ID NO:5) comprising 20 to 26 moles of SA per mole CD80 ECD-Fc fusion protein. In certain aspects a pharmaceutical composition comprises CD80 ECD-Fc fusion proteins (e.g. comprising SEQ ID NO:5) comprising 30 to 40 moles of SA per mole CD80 ECD-Fc fusion protein. In certain aspects a pharmaceutical composition comprises CD80 ECD-Fc fusion proteins (e.g. comprising SEQ ID NO:5) comprising 10, 15, 20, 26, 30, 35, or 40 moles of SA per mole CD80 ECD-Fc fusion protein. In certain aspects a pharmaceutical composition comprises CD80 ECD-Fc fusion proteins (e.g. comprising SEQ ID NO:5) comprising 10, 15, 20, 25, 30, 35, or 40 moles of SA per mole CD80 ECD-Fc fusion protein. In certain aspects a pharmaceutical composition comprises CD80 ECD-Fc fusion proteins (e.g. comprising SEQ ID NO:5) comprising at least 15 moles of SA per mole CD80 ECD-Fc fusion protein. In certain aspects a pharmaceutical composition comprises CD80 ECD-Fc fusion proteins (e.g. comprising SEQ ID NO:5) comprising at least 20 moles of SA per mole CD80 ECD-Fc fusion protein. In certain aspects a pharmaceutical composition comprises CD80 ECD-Fc fusion proteins (e.g. comprising SEQ ID NO:5) comprising at least 25 moles of SA per mole CD80 ECD-Fc fusion protein. In certain aspects a pharmaceutical composition comprises CD80 ECD-Fc fusion proteins (e.g. comprising SEQ ID NO:5) comprising at least 26 moles of SA per mole CD80 ECD-Fc fusion protein. In certain aspects a pharmaceutical composition comprises CD80 ECD-Fc fusion proteins (e.g. comprising SEQ ID NO:5) comprising at least 30 moles of SA per mole CD80 ECD-Fc fusion protein. In certain aspects a pharmaceutical composition comprises CD80 ECD-Fc fusion proteins (e.g. comprising SEQ ID NO:5) comprising at least 35 moles of SA per mole CD80 ECD-Fc fusion protein. In certain aspects a pharmaceutical composition comprises CD80 ECD-Fc fusion proteins (e.g. comprising SEQ ID NO:5) comprising at least 40 moles of SA per mole CD80 ECD-Fc fusion protein.

### 4. Methods and Uses of CD80 Extracellular Domain Fc Fusion Proteins

Presented herein are methods for treating a solid tumor in a human subject comprising administering to a subject in need thereof a CD80 ECD-Fc fusion protein. The CD80 ECD-Fc fusion protein can comprise the extracellular domain of human CD80 and the Fe domain of human IgG1.

In one aspect, a method of treating a solid tumor in a human patient comprises administering to the patient about 1,260 mg of a CD80 ECD fusion protein (e.g., comprising the amino acid sequence set forth in SEQ ID NO:5), e.g., once every three weeks. In one aspect, a method of treating a solid tumor in a human patient comprises administering to the patient about 840 mg of a CD80 ECD fusion protein (e.g., comprising the amino acid sequence set forth in SEQ ID NO:5), e.g., once every three weeks. In one aspect, a method of treating a solid tumor in a human patient comprises administering to the patient about 700 mg of a CD80 ECD fusion protein (e.g., comprising the amino acid sequence set forth in SEQ ID NO:5), e.g., once every three weeks. In one aspect, a method of treating a solid tumor in a human patient comprises administering to the patient about 630 mg of a CD80 ECD fusion protein (e.g., comprising the amino acid sequence set forth in SEQ ID NO:5), e.g., once every three weeks. In one aspect, a method of treating a solid tumor in a human patient comprises administering to the patient about 560 mg of a CD80 ECD fusion protein (e.g., comprising the amino acid sequence set forth in SEQ ID NO:5), e.g., once every three weeks. In one aspect, a method of treating a solid tumor in a human patient comprises administering to the patient about 420 mg of a CD80 ECD fusion protein (e.g., comprising the amino acid sequence set forth in SEQ ID NO:5), e.g., once every three weeks. In one aspect, a method of treating a solid tumor in a human patient comprises administering to the patient about 280 mg of a CD80 ECD fusion protein (e.g., comprising the amino acid sequence set forth in SEQ ID NO:5), e.g., once every three weeks. In one aspect, a method of treating a solid tumor in a human patient comprises administering to the patient about 210 mg of a CD80 ECD fusion protein (e.g., comprising the amino acid sequence set forth in SEQ ID NO:5), e.g., once every three weeks. In one aspect, a method of treating a solid tumor in a human patient comprises administering to the patient about 140 mg of a CD80 ECD fusion protein (e.g., comprising the amino acid sequence set forth in SEQ ID NO:5), e.g., once every three weeks.

In one aspect, a method of treating a solid tumor in a human patient comprises administering to the patient 1,260 mg of a CD80 ECD fusion protein (e.g., comprising the amino acid sequence set forth in SEQ ID NO:5), e.g., once every three weeks. In one aspect, a method of treating a solid tumor in a human patient comprises administering to the patient 840 mg of a CD80 ECD fusion protein (e.g., comprising the amino acid sequence set forth in SEQ ID NO:5), e.g., once every three weeks. In one aspect, a method of treating a solid tumor in a human patient comprises administering to the patient 700 mg of a CD80 ECD fusion protein (e.g., comprising the amino acid sequence set forth in SEQ ID NO:5), e.g., once every three weeks. In one aspect, a method of treating a solid tumor in a human patient comprises administering to the patient 630 mg of a CD80 ECD fusion protein (e.g., comprising the amino acid sequence set forth in SEQ ID NO:5), e.g., once every three weeks. In one aspect, a method of treating a solid tumor in a human patient comprises administering to the patient 560 mg of a CD80 ECD fusion protein (e.g., comprising the amino acid sequence set forth in SEQ ID NO:5), e.g., once every three weeks. In one aspect, a method of treating a solid tumor in a human patient comprises administering to the patient 420 mg of a CD80 ECD fusion protein (e.g., comprising the amino acid sequence set forth in SEQ ID NO:5), e.g., once every three weeks. In one aspect, a method of treating a solid tumor in a human patient comprises administering to the patient 280 mg of a CD80 ECD fusion protein (e.g., comprising the amino acid sequence set forth in SEQ ID NO:5), e.g., once every three weeks. In one aspect, a method of treating a solid tumor in a human patient comprises administering to the patient 210 mg of a CD80 ECD fusion protein (e.g., comprising the amino acid sequence set forth in SEQ ID NO:5), e.g., once every three weeks. In one aspect, a method of treating a solid tumor in a human patient comprises administering to the patient 140 mg of a CD80 ECD fusion protein (e.g., comprising the amino acid sequence set forth in SEQ ID NO:5), e.g., once every three weeks.

In one aspect, a method of treating a solid tumor in a human patient comprises administering to the patient about 140 mg to about 1,260 mg of a CD80 ECD fusion protein (e.g., comprising the amino acid sequence set forth in SEQ ID NO:5), e.g., once every three weeks. In one aspect, a method of treating a solid tumor in a human patient comprises administering to the patient about 280 mg to about 1,260 mg of a CD80 ECD fusion protein (e.g., comprising the amino acid sequence set forth in SEQ ID NO:5), e.g., once every three weeks. In one aspect, a method of treating a solid tumor in a human patient comprises administering to the patient about 140 mg to about 700 mg of a CD80 ECD fusion protein (e.g., comprising the amino acid sequence set forth in SEQ ID NO:5), e.g., once every three weeks. In one aspect, a method of treating a solid tumor in a human patient comprises administering to the patient about 280 mg to about 700 mg of a CD80 ECD fusion protein (e.g., comprising the amino acid sequence set forth in SEQ ID NO:5), e.g., once every three weeks. In one aspect, a method of treating a solid tumor in a human patient comprises administering to the patient about 140 mg to about 210 mg of a CD80 ECD fusion protein (e.g., comprising the amino acid sequence set forth in SEQ ID NO:5), e.g., once every three weeks. In one aspect, a method of treating a solid tumor in a human patient comprises administering to the patient about 210 mg to about 280 mg of a CD80 ECD fusion protein (e.g., comprising the amino acid sequence set forth in SEQ ID NO:5), e.g., once every three weeks. In one aspect, a method of treating a solid tumor in a human patient comprises administering to the patient about 280 mg to about 420 mg of a CD80 ECD fusion protein (e.g., comprising the amino acid sequence set forth in SEQ ID NO:5), e.g., once every three weeks. In one aspect, a method of treating a solid tumor in a human patient comprises administering to the patient about 420 mg to about 560 mg of a CD80 ECD fusion protein (e.g., comprising the amino acid sequence set forth in SEQ ID NO:5), e.g., once every three weeks. In one aspect, a method of treating a solid tumor in a human patient comprises administering to the patient about 560 mg to about 630 mg of a CD80 ECD fusion protein (e.g., comprising the amino acid sequence set forth in SEQ ID NO:5), e.g., once every three weeks. In one aspect, a method of treating a solid tumor in a human patient comprises administering to the patient about 630 mg to about 700 mg of a CD80 ECD fusion protein (e.g., comprising the amino acid sequence set forth in SEQ ID NO:5), e.g., once every three weeks. In one aspect, a method of treating a solid tumor in a human patient comprises administering to the patient about 700 mg to about 840 mg of a CD80 ECD fusion protein (e.g., comprising the amino acid sequence set forth in SEQ ID NO:5), e.g., once every three weeks. In one aspect, a method of treating a solid tumor in a human patient comprises administering to the patient about 840 mg to about 1,260 mg of a CD80 ECD fusion protein (e.g., comprising the amino acid sequence set forth in SEQ ID NO:5), e.g., once every three weeks.

According to the methods provided herein, a CD80 ECD fusion protein (e.g., comprising the amino acid sequence set forth in SEQ ID NO:5) can be administered intravenously.

According to the methods provided herein, the solid tumor can be, for example, an advanced solid tumor. In certain instances, the solid tumor is not a primary central nervous system tumor.

In certain instances, the solid tumor is a renal cell carcinoma.

In certain instances, the solid tumor is a melanoma.

In certain instances, the solid tumor is a colorectal cancer, breast cancer, gastric cancer, non-small cell lung cancer, small cell lung cancer, melanoma, squamous cell carcinoma of the head and neck, ovarian cancer, pancreatic cancer, renal cell carcinoma, hepatocellular carcinoma, bladder cancer, or endometrial cancer.

In certain instances, the solid tumor is a sarcoma.

The patient to be treated according to the methods provided herein may have received prior therapy with at least one PD-1/PD-L1 antagonist selected from a PD-1 antagonist and a PD-L1 antagonist. The PD-1/PD-L1 antagonist can be, for example, nivolumab, pembrolizumab, atezolizumab, durvalumab, or avelumab. The PD-1/PDL-1 antagonist may have been administered in an advanced or metastatic setting. In other instances, the patient to be treated according to the methods provided herein has not received prior therapy with a PD-1/PDL-1 antagonist.

The patient to be treated according to the methods provided herein may have received prior therapy with an anti-angiogenic agent. The anti-angiogenic agent can be, for example, sunitinib, sorafenib, pazopanib, axitinib, tivozanib, ramucirumab, or bevacizumab. The anti-angiogenic agent may have been administered in an advanced or metastatic setting.

The patient to be treated according to the methods provided herein, for example a patient with a melanoma, may have a BRAF mutation. The patient may have received prior therapy with a BRAF inhibitor. The BRAF inhibitor can be, for example, vemurafenib and dabrafenib. The BRAF inhibitor may have been administered in an advanced or metastatic setting.

The tumor to be treated according to the methods provided herein can be recurrent or progressive after a therapy selected from surgery, chemotherapy, radiation therapy, and a combination thereof.

The tumor to be treated according to the methods provided herein can be resistant or non-responsive to a PD-1/PD-L1 antagonist, such as nivolumab, pembrolizumab, atezolizumab, durvalumab, or avelumab. The tumor to be treated according to the methods provided herein can be resistant or non-responsive to an anti-angiogenic agent, such as sunitinib, sorafenib, pazopanib, axitinib, tivozanib, ramucirumab, or bevacizumab. The tumor to be treated according to the methods provided herein can be resistant or non-responsive to a BRAF inhibitor, such as vemurafenib or dabrafenib.

The tumor to be treated according to the methods provided herein can be refractory to a PD-1/PD-L1 antagonist, such as nivolumab, pembrolizumab, atezolizumab, durvalumab, or avelumab. The tumor to be treated according to the methods provided herein can be refractory to an anti-angiogenic agent, such as sunitinib, sorafenib, pazopanib, axitinib, tivozanib, ramucirumab, or bevacizumab. The tumor to be treated according to the methods provided herein can be refractory to a BRAF inhibitor, such as vemurafenib or dabrafenib.

In certain instances, the solid tumor is a sarcoma.

The tumor to be treated according to the methods provided herein can be recurrent after treatment with a PD-1/PD-L1 antagonist, such as nivolumab, pembrolizumab, atezolizumab, durvalumab, or avelumab. The tumor to be treated according to the methods provided herein can be recurrent after treatment with an anti-angiogenic agent, such as sunitinib, sorafenib, pazopanib, axitinib, tivozanib, ramucirumab, or bevacizumab. The tumor to be treated according to the methods provided herein can be recurrent after treatment with a BRAF inhibitor, such as vemurafenib or dabrafenib.

In some embodiments, the present invention relates to a CD80 ECD-Fc fusion protein or pharmaceutical composition provided herein for use as a medicament for the treatment of a solid tumor, wherein the medicament is for administration at 140 mg to 1,260 mg (e.g., 140 mg, 210 mg, 280 mg, 420 mg, 560 mg, 630 mg, 700 mg, 840 mg, or 1,260 mg) of the CD80 ECD-Fc fusion, e.g., once every three weeks. In some aspects, the present invention relates to an CD80 ECD-Fc fusion protein or pharmaceutical composition provided herein, for use in a method for the treatment of a solid tumor wherein 140 mg to 1,260 mg (e.g., 140 mg, 210 mg, 280 mg, 420 mg, 560 mg, 630 mg, 700 mg, 840 mg, or 1,260 mg) of the CD80 ECD-Fc fusion is administered, e.g., once every three weeks.

In some embodiments, the present invention relates to a CD80 ECD-Fc fusion protein or pharmaceutical composition provided herein for use as a medicament for the treatment of a solid tumor, wherein the medicament is for administration at 140 mg to 700 mg (e.g., 140 mg, 210 mg, 280 mg, 420 mg, 560 mg, 630 mg, or 700 mg) of the CD80 ECD-Fc fusion, e.g., once every three weeks. In some aspects, the present invention relates to an CD80 ECD-Fc fusion protein or pharmaceutical composition provided herein, for use in a method for the treatment of a solid tumor wherein 140 mg to 700 mg (e.g., 140 mg, 210 mg, 280 mg, 420 mg, 560 mg, 630 mg, or 700 mg) of the CD80 ECD-Fc fusion is administered, e.g., once every three weeks.

### EXAMPLES

The examples discussed below are intended to be purely exemplary of the invention and should not be considered to limit the invention in any way. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1: Cytokine Release Effects of a CD80 ECD-Fc Fusion Molecule

### Methods

Protein Treatments

A human CD80 ECD IgG1 Fc fusion protein (“CD80-Fc”) was bound to magnetic protein-A beads (Life Technologies) in T-cell proliferation media containing RPMI 1640, 100 IU Penicillin/100 ug/ml Streptomycin, 2 mM L-Glutamine, 100 nM non-essential amino acids, 55 uM 2-mercaptoethanol and 10% ultra low-IgG fetal bovine serum. Binding reactions were carried out in 96-well flat-bottom tissue culture plates at a volume of 100 μl per well with a bead concentration of 3 million beads per ml. CD80-Fc was bound to the beads across a series of concentrations: 10, 1, 0.1 μg/ml. An additional set of binding reactions was also performed with the addition of 3 ng/ml OKT3-scFv. Proteins were allowed to bind for 1 hour at room temperature on a rocking platform, following which 100 μl of 20 μg/ml (final concentration 10 μg/ml) IgG1 Free-Fc (FPT) was added to each well and allowed to bind for an additional hour in order to block any unoccupied Protein-A binding sites on the beads. The fully loaded and blocked beads were then washed 3 times with PBS using a magnetic 96-well plate stand in order to remove unbound proteins. 100 μl of Human Pan T-cells at a concentration of 1×10⁶ cells/ml was then added to each well of dry, washed beads. Each condition was tested in triplicate.

Cells

Human peripheral blood mononuclear cells (PBMCs) were isolated from apheresis-enriched blood (buffy coats) collected from healthy donors ˜18 hrs prior to isolation using Ficoll® (Biochrom) gradient density centrifugation. Pan T-cells were then isolated from PBMCs using a Human Pan T-cell isolation kit (Miltenyi). T-cells were seeded at a density of 1 million cells/ml in T225 tissue culture flasks in proliferation media (above) supplemented with 8 ng/ml IL-2 and Human T-cell Activator Dynabeads® (Life Tech) 1 bead/cell. Following seeding, cells were fed with fresh IL-2 and continually kept at a concentration of 0.3 million cells/ml by the addition of fresh proliferation media every 2 days. Cells were kept in a 37° C. water-jacketed incubator maintained at 5% CO₂. After 6 days of expansion, the activator-beads were removed using a magnetic tube stand and the cells were resuspended at a concentration of 1 million cells/ml in fresh proliferation media without IL-2. 24 hours later the cells were put into assay with Protein-A bead immobilized proteins.

Cytokine Measurements

Soluble Interferon Gamma (IFN-γ) and Tumor Necrosis Factor Alpha (TNF-α) levels were measured in the supernatants using HTRF-ELISA kits (Cisbio) 24 hours after the cells had been treated with the Protein-A bead immobilized proteins according to the manufacturer's instructions.

### Results

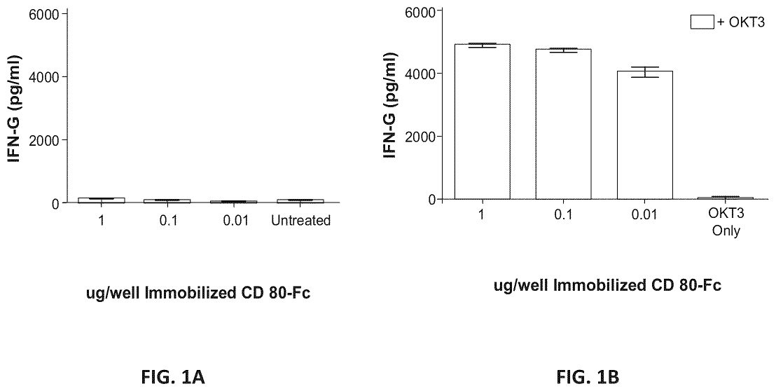

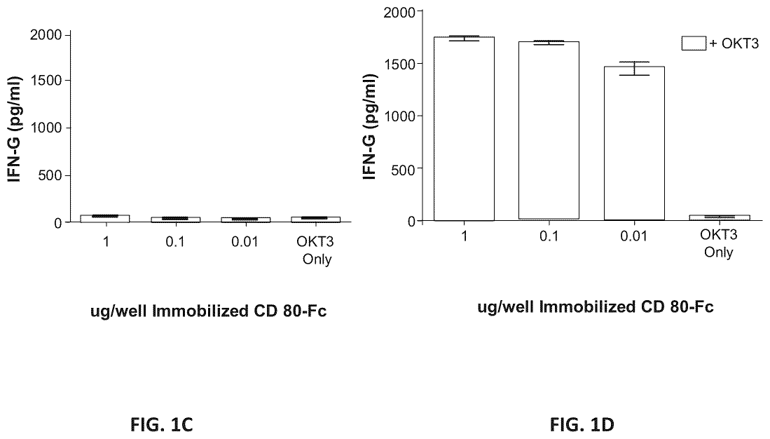

Bead-immobilized CD80-Fc alone did not cause significant human T-cell activation, as measured by soluble cytokine production (FIGS. 1a & c). However, when a small amount of OKT3-scFv was immobilized along with CD80-Fc, robust CD80-dependent IFN-γ and TNF-α release was observed (FIGS. 1b & d). The amount of OKT3-scFv used here was too low to cause T-cell stimulation on its own and therefore required the presence of CD80 as a co-stimulatory protein. These results therefore confirm the CD80-Fc used in this assay was indeed biologically active.

While release of IFN-γ and TNF-α in this assay showed that the CD80-Fc was biologically active, an excessive release of cytokines such as IFN-γ and TNF-α can be harmful. Thus, to address the potential safety of CD80 ECD-Fc treatment, these results were compared to earlier published results with TGN1412, a monocolonal anti-CD28 antibody that was shown to be a T-cell “superagonist” and to release excessive and harmful levels of cytokines such as IFN-γ and TNF-α in human subjects.

Immobilized TGN1412 alone appears to be significantly more potent at inducing cytokine release from human T-cells than human CD80 alone. Findlay et al., J. Immunological Methods 352: 1-12 (2010), reported that 1 μg/well of TGN1412 caused robust TNFα release, ˜2,000 pg/ml, and Vessillier et al., J. Immunological Methods 424: 43-52 (2015), reported the same amount of TGN1412 caused robust IFN-γ, ˜10,000 pg/ml. The same amount of immobilized CD80-Fc did not cause significant release of either cytokine. These results suggest that CD80-Fc is at least 1000-fold less potent at inducing cytokine release compared to TGN1412 and therefore poses a significantly lower risk of inducing cytokine storm in humans than TGN1412.

### Example 2: Effects of a CD80 ECD-Fc Fusion Molecule on CT26 Tumors In Vivo with Fc Domains with Different Sialic Acid (SA) Content

An in vivo study was conducted in CT26 tumors to analyze the effects of three different lots of CD80 ECD fused to wild-type human IgG1 Fc having different sialic acid (SA) contents. Specifically, lot E of the CD80 ECD-Fc contains 20 mol SA/mol protein, lot D contains 15 mol SA/mol protein, and lot A contains 5 mol SA/mol protein.

Seven-week-old female BALB/c mice were purchased from Charles River Laboratories (Hollister, Calif.) and were acclimated for one week before the study was initiated. The murine colorectal carcinoma cell line CT26 was implanted subcutaneously over the right flank of the mice at 1.0×10⁶ cells/200 l/mouse. Prior to inoculation, the cells were cultured for no more than three passages in RPMI 1640 medium supplemented with 10% heat-inactivated Fetal Bovine Serum (FBS), 2 mM L-Glutamine. Cells were grown at 37° C. in a humidified atmosphere with 5% CO₂. Upon reaching 80-85% confluence, cells were harvested and resuspended in a 1:1 mixture of serum-free RPMI 1640 and Matrigel® at 5×10⁶ cells per milliliter.

Mice were monitored for tumor growth twice weekly following cell implantation. For tumor measurements, the length and width of each tumor was measured using calipers and volume was calculated according to the formula: tumor volume (mm³)=(width (mm)×length (mm)) 2/2. On Day 7, all tumors were measured, and mice were randomly assigned to seven treatment groups (n=10 mice per experimental group). The mean tumor volume for all animals enrolled was 94 mm³. The first group was injected with 200 μl of PBS (control) intravenously (i.v.) into the tail vein. The second group was injected with CD80 ECD-Fc at 20 mol SA/mol protein (lot E) i.v. dosed at 0.3 mg/kg. The third group was injected with CD80 ECD-Fc at 20 mol SA/mol protein (lot E) i.v. dosed at 0.6 mg/kg. The fourth group was injected with CD80 ECD-Fc at 15 mol SA/mol protein (lot D) i.v. dosed at 0.3 mg/kg. The fifth group was injected with CD80 ECD-Fc at 15 mol SA/mol protein (lot D) i.v. dosed at 0.6 mg/kg. The sixth group was injected with CD80 ECD-Fc at 5 mol SA/mol protein (lot A) i.v. dosed at 0.3 mg/kg. The seventh group was injected with CD80 ECD-Fc at 5 mol SA/mol protein (lot A) i.v. dosed at 0.6 mg/kg. Tumors were measured on day 10, 14, 16, 18, 22, 24.

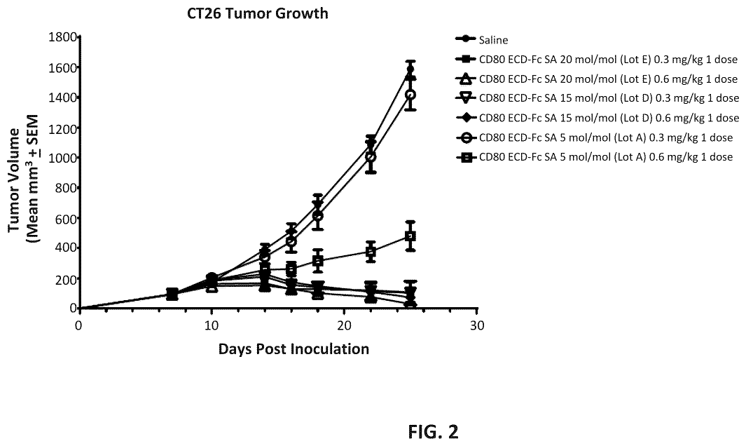

Treatment with CD80 ECD-Fc at 20 mol SA/mol protein (lot E) dosed at 0.3 or 0.6 mg/kg resulted in a 93% and 98% inhibition of tumor growth compared to the control (P<0.001). Treatment with CD80 ECD-Fc at 15 mol SA/mol protein (lot D) dosed at 0.3 or 0.6 mg/kg resulted in a 93% and 95% inhibition of tumor growth compared to the control (p<0.001). By comparison, treatment with CD80 ECD-Fc lot A at 0.3 mg/kg (with 5 mol SA/mol protein) did not inhibit tumor growth compared to the control and when dosed at 0.6 mg/kg it only induced 70% inhibition (p<0.001) (FIG. 2).

The incidence of tumor-free mice was analyzed at day 37. Treatment with CD80 ECD-Fc at 20 mol/mol SA (lot E) dosed at 0.3 or 0.6 mg/kg led to complete tumor regression in 8/10 (80%) or 10/10 (100%) of the mice. Treatment with CD80 ECD-Fc at 15 mol/mol SA (lot D) dosed at 0.3 or 0.6 mg/kg led to complete tumor regression in 9/10 (90%) of the mice. By comparison, treatment with CD80 ECD-Fc lot A dosed at 0.6 mg/kg induced tumor regression only in 1/10 (10%) of the mice, as shown in Table 1 below.

TABLE 1
Sialic Acid Content and Anti-Tumor Activity

| Treatment group | Tumor-free mice at day 37 | |
|---|---|---|
| Saline | 0% | (0/10 mice) |
| CD80 ECD-Fc SA 20 mol/mol (lot E) at 0.3 mg/kg 1 dose | 80% | (8/10 mice) |
| CD80 ECD-Fc SA 20 mol/mol (lot E) at 0.6 mg/kg 1 dose | 100% | (10/10 mice) |
| CD80 ECD-Fc SA 15 mol/mol (lot D) at 0.3 mg/kg 1 dose | 90% | (9/10 mice) |
| CD80 ECD-Fc SA 15 mol/mol (lot D) at 0.6 mg/kg 1 dose | 90% | (9/10 mice) |
| CD80 ECD-Fc SA 5 mol/mol (lot A) at 0.3 mg/kg 1 dose | 0% | (0/10 mice) |
| CD80 ECD-Fc SA 5 mol/mol (lot A) at 0.6 mg/kg 1 dose | 10% | (1/10 mice) |

### Example 3: Effects of a Murine CD80 ECD—Murine Fc Fusion Molecule on Tumor Growth in Three Different Syngeneic Tumor Models

In vivo studies were conducted using a mouse surrogate comprising the extracellular domain (ECD) of murine CD80 linked to the Fc domain of mouse IgG2a wild type (murine CD80 ECD-Fc). The effects of murine CD80 ECD-Fc were compared with those of the anti-CTLA4 antibody clone 9D9 (IgG2b) in three different syngeneic tumor models: the CT26 colon carcinoma, the MC38 colon carcinoma and the B16 melanoma models.

### CT26 Tumor Model

Seven-week-old female BALB/c mice were purchased from Charles River Laboratories (Hollister, Calif.) and were acclimated for one week before the study was initiated. The murine colorectal carcinoma cell line CT26 was implanted subcutaneously over the right flank of the mice at 1.0×10⁶ cells/200 l/mouse. Prior to inoculation, the cells were cultured for no more than three passages in RPMI 1640 medium supplemented with 10% heat-inactivated Fetal Bovine Serum (FBS), 2 mM L-Glutamine. Cells were grown at 37° C. in a humidified atmosphere with 5% CO₂. Upon reaching 80-85% confluence, cells were harvested and resuspended in a 1:1 mixture of serum-free RPMI 1640 and matrigel.

Mice were monitored twice weekly following cell implantation for tumor growth. For tumor measurements, the length and width of each tumor was measured using calipers and volume was calculated according to the formula: tumor volume (mm³)=(width (mm)×length (mm)) 2/2. On Day 7, all tumors were measured, and mice were randomly assigned to seven treatment groups (n=15 mice per experimental group). The mean tumor volume for all animals enrolled was 96 mm³. Mice were dosed 3 times: on day 4, 7, and 11. The first group was injected with mouse IgG2b (mIgG2b) i.p. dosed at 10 mg/kg (control). The second group was injected with murine CD80 ECD-Fc 20 mol/mol SA i.v. dosed at 0.3 mg/kg. The third group was injected with anti-CTLA4 antibody clone 9D9 (IgG2b) i.p. dosed at 1.5 mg/kg. The fourth group was injected with anti-CTLA4 antibody clone 9D9 (IgG2b) i.p. dosed at 10 mg/kg. Tumors were measured on days 10, 13, 17, 19, 21, and 24.

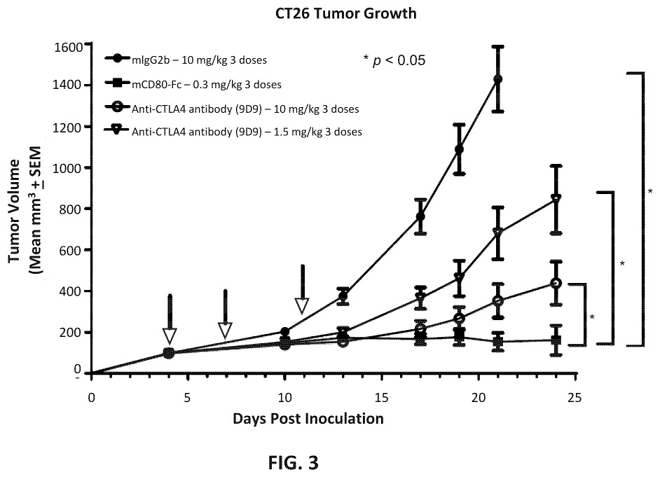

At day 21 (when all the controls were still in the study), treatment with murine CD80 ECD-Fc at 20 mol/mol SA dosed at 0.3 mg/kg resulted in 90% inhibition of tumor growth compared to the control (p<0.001). Treatment with anti-CTLA4 antibody at 10 mg/kg resulted in 75% inhibition of tumor growth compared to the control (P<0.001). By comparison, treatment with anti-CTLA4 antibody at 1.5 mg/kg only resulted in 53% inhibition of tumor growth (P<0.001) (FIG. 3). At day 21, the impact of treatment with murine CD80 ECD-Fc at 20 mol/mol SA dosed at 0.3 mg/kg on tumor growth was significantly greater than anti-CTLA4 antibody dosed at 1.5 mg/kg (p<0.001) or at 10 mg/kg (p=0.009).

The incidence of tumor-free mice was analyzed at day 37. Treatment with murine CD80 ECD-Fc at 20 mol/mol SA dosed at 0.3 mg/kg led to complete tumor regression in 7/15 (47%) of the mice. Treatment with anti-CTLA4 antibody at 10 mg/kg led to complete tumor regression in 3/15 (20%) of the mice. None of the mice treated with anti-CTLA4 antibody at 1.5 mg/kg had complete tumor regression.

### MC38 Tumor Model

Seven-week-old female C57Bl/6 mice were purchased from Charles River Laboratories (Hollister, Calif.) and were acclimated for one week before the study was initiated. The murine colorectal carcinoma cell line MC38 was implanted subcutaneously over the right flank of the mice at 0.5×10⁶ cells/100 l/mouse. Prior to inoculation, the cells were cultured for no more than three passages in RPMI 1640 medium supplemented with 10% heat-inactivated Fetal Bovine Serum (FBS), 2 mM L-Glutamine. Cells were grown at 37° C. in a humidified atmosphere with 5% CO₂. Upon reaching 80-85% confluence, cells were harvested and resuspended in a 1:1 mixture of serum-free RPMI 1640 and matrigel.

Mice were monitored twice weekly following cell implantation for tumor growth. For tumor measurements, the length and width of each tumor was measured using calipers and volume was calculated according to the formula: tumor volume (mm³)=(width (mm)×length (mm)) 2/2. On Day 7, all tumors were measured, and mice were randomly assigned to seven treatment groups (n=15 mice per experimental group). The mean tumor volume for all animals enrolled was 78 mm³. Mice were dosed 3 times: on day 7, 10, and 14. The first group was injected with mouse IgG2b (mIgG2b) i.p. dosed at 10 mg/kg (control). The second group was injected with murine CD80 ECD-Fc 20 mol/mol SA i.v. dosed at 3 mg/kg. The third group was injected with anti-CTLA4 antibody clone 9D9 (IgG2b) i.p. dosed at 1.5 mg/kg. The fourth group was injected with anti-CTLA4 antibody clone 9D9 (IgG2b) i.p. dosed at 10 mg/kg. Tumors were measured on days 11, 14, 17, and 19.

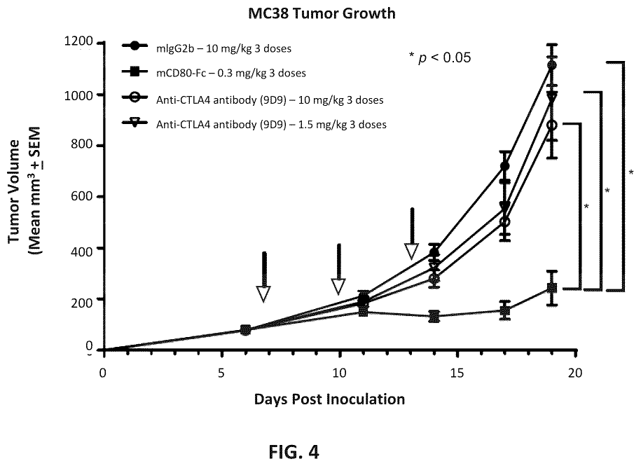

At day 19 (when all the controls were still in the study), treatment with murine CD80 ECD-Fc at 20 mol/mol SA dosed at 3 mg/kg resulted in 79% inhibition of tumor growth compared to the control (P<0.001). Moreover, murine CD80 ECD-Fc at 20 mol/mol SA had a greater impact on tumor growth compared to anti-CTLA4 antibody (P<0.001). Treatment with anti-CTLA4 antibody at 10 mg/kg reduced tumor growth by 21% compared to the control (P=0.05) while at 1.5 mg/kg did not significantly affect tumor size (FIG. 4). At day 21, the impact of treatment with murine CD80 ECD-Fc at 20 mol/mol SA dosed at 3 mg/kg on tumor growth was significantly greater than anti-CTLA4 antibody dosed at 1.5 mg/kg (P<0.001) or at 10 mg/kg (P=0.009).

While a 3 mg/kg dose of CD80 ECD-Fc was used for these experiments, a 0.3 mg/kg dose of CD80 ECD-Fc also reduced tumor cell growth in the MC38 tumor model).

### B16 Tumor Model

Seven-week-old female C57Bl/6 mice were purchased from Charles River Laboratories (Hollister, Calif.) and were acclimated for one week before the study was initiated. The murine melanoma cell line B16-F10 was implanted subcutaneously over the right flank of the mice at 0.5×10⁶ cells/100 μl/mouse. Prior to inoculation, the cells were cultured for no more than three passages in DMEM medium supplemented with 10% heat-inactivated Fetal Bovine Serum (FBS), 2 mM L-Glutamine. Cells were grown at 37° C. in a humidified atmosphere with 5% CO₂. Upon reaching 80-85% confluence, cells were harvested and resuspended in a 1:1 mixture of serum-free DMEM and matrigel.

Mice were monitored twice weekly following cell implantation for tumor growth. For tumor measurements, the length and width of each tumor was measured using calipers and volume was calculated according to the formula: tumor volume (mm³)=(width (mm)×length (mm)) 2/2. On Day 7, all tumors were measured, and mice were randomly assigned to seven treatment groups (n=15 mice per experimental group). The mean tumor volume for all animals enrolled was 70 mm³. Mice were dosed 3 times: on day 3, 6 and 10. The first group was injected with mouse IgG2b (mIgG2b) dosed i.p. at 10 mg/kg (control). The second group was injected with murine CD80 ECD-Fc 20 mol/mol SA i.v. dosed at 3 mg/kg. The third group was injected with anti-CTLA4 antibody clone 9D9 (IgG2b) i.p. dosed at 1.5 mg/kg. The fourth group was injected with anti-CTLA4 antibody clone 9D9 (IgG2b) i.p. dosed at 10 mg/kg. Tumors were measured on days 10, 13, 15, 16, 17.

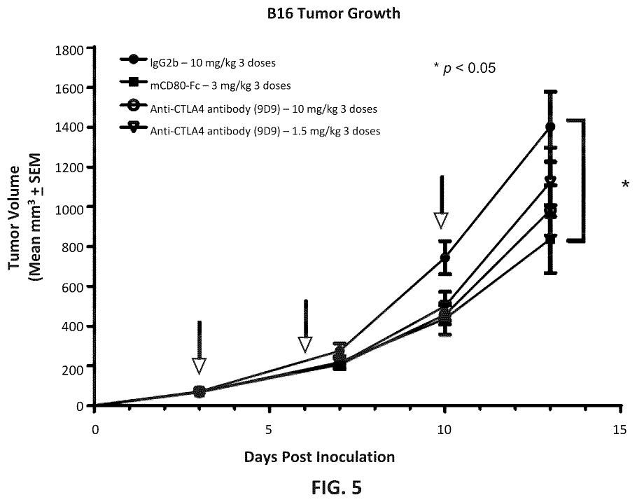

At day 13 (when all the controls were still in the study) treatment with murine CD80 ECD-Fc at 20 mol/mol SA dosed at 3 mg/kg resulted in 41% inhibition of tumor growth compared to the control (P<0.001). Treatment with anti-CTLA4 antibody at 10 mg/kg or 1.5 mg/kg did not significantly affect tumor growth compared to the control (FIG. 5).

Additional experiments showed that murine CD80 ECD-Fc exerts anti-tumor activity in EMT6 (breast cancer), A20 (reticulum cell carcinoma) and WEH164 tumor models.

Mice that rejected tumors in response to murine CD80 ECD-Fc were protected from subsequent rechallenge.

### Example 4: Interaction of CD80 and PD-L1

CD80 has been reported to interact with 3 binding partners: CD28, CTLA-4, and PD-L1. Binding studies were performed to determine the relevant binding partners of a human CD80 ECD:human IgG Fc fusion protein comprising the amino acid sequence of SEQ ID NO:5 (i.e., hCD80ECD:hIgG1Fc). These studies used surface plasmon resonance (SPR), enzyme-linked immunosorbent assay (ELISA), and flow cytometry.

The SPR studies demonstrated that hCD80ECD:hIgG1Fc has the highest affinity for CTLA-4 (1.8 nM), moderate affinity for PD-L1 (183 nM), and low affinity for CD28 (>1 μM). The low affinity of hCD80ECD:hIgG1Fc for CD28 is consistent with literature reports. (See Greene et al., Journal of Biological Chemistry 271: 26762-26771 (1996) and Collins et al., Immunity 17: 201-201 (2002).)

Results from an ELISA study also supported the strong affinity of hCD80ECD:hIgG1Fc for CTLA-4, and flow cytometry studies showed engagement of hCD80ECD:hIgG1Fc with cell surface CTLA-4 and CD28 but not PD-L1. When hCD80ECD:hIgG1Fc binding was tested on human peripheral blood mononuclear cells (PBMCs), hCD80ECD:hIgG1Fc primarily bound to T-cell subsets in a concentration-dependent manner. Potent binding was also demonstrated with in vitro-activated conventional CD4+ T-cells and T_{reg}. HCD80ECD:hIgG1Fc binding to T-cells was mediated via CD28 and CTLA-4; no binding to cell-surface PD-L1 could be demonstrated, in contrast to the cell-free SPR studies.

Thus, the biological significance of the CD80 interaction with PD-L1 is not clear.

### Example 5: Nonclinical Pharmacokinetics

The pharmacokinetics (PK) and toxicokinetics (TK) of hCD80ECD:hIgG1Fc were investigated in mice, rats, and cynomolgus monkeys. These studies included 1 single-dose PK study in mice that examined doses of hCD80ECD:hIgG1Fc from 0.03 mg/kg to 3 mg/kg and 2 repeat-dose studies with 4-weekly dosing each in rats and cynomolgus monkeys that examined doses of hCD80ECD:hIgG1Fc from 1 mg/kg to 100 mg/kg. Among 4 repeat-dose studies, there was 1 PK study in rats, 1 pilot toxicology study in cynomolgus monkeys, and 1 Good Laboratory Practice (GLP) toxicology study in each species. In all studies, hCD80ECD:hIgG1Fc was administered by intravenous (IV) administration.

Following single IV dose ranging from 0.03 to 3 mg/kg in mice, the maximum observed serum concentration (Cₘₐₓ) of hCD80ECD:hIgG1Fc increased more than dose proportionally from 0.03 mg/kg to 0.9 mg/kg and dose proportionally from 0.9 mg/kg to 3 mg/kg. The area under serum concentration (AUC)-time curve from day 0 to day 4 increased in a dose-proportional manner from 0.03 mg/kg to 3 mg/kg with estimated clearance of 18.0 to 26.3 mL/day/kg and terminal half-life of 1-2 days. In the 4-week repeat weekly dosing studies in rats or cynomolgus monkeys, both Cₘₐₓ and the AUC-time curve from day 0 to day 7 increased approximately in proportion with dose level in the dose range from 1 mg/kg to 100 mg/kg following the first and fourth doses. The estimated terminal half-life was 4 to 6 days. Following 4-weekly dose administration, there was little to no accumulation. Anti-drug antibodies (ADA) were present in the majority of rats (11/16 and 23/24 for the PK study and the GLP toxicology study, respectively). Seven out of 12 and 2 out of 30 cynomolgus monkeys treated with hCD80ECD:hIgG1Fc from the pilot toxicology study and the GLP toxicology study, respectively, were ADA-positive. The impact of ADA on the serum concentration of hCD80ECD:hIgG1Fc was observed and highly variable in ADA-positive animals.

In summary, hCD80ECD:hIgG1Fc has linear clearance for the dose range from 0.03 mg/kg to 3 mg/kg in mice and from 1 mg/kg to 100 mg/kg in rats and cynomolgus monkeys. HCD80ECD:hIgG1Fc has faster clearance and shorter half-life than a typical monoclonal antibody (mAb) in animals. The PK characteristics of hCD80ECD:hIgG1Fc in animals support IV infusion in humans.

### Example 6: Toxicology

Toxicology studies were also performed with hCD80ECD:hIgG1Fc. These studies include a pilot repeat-dose toxicity study in cynomolgus monkeys and Investigational New Drug (IND) application-enabling GLP repeat-dose toxicity studies in rats and cynomolgus monkeys.

In the repeat-dose GLP toxicology studies in rats, hCD80ECD:hIgG1Fc was administered at dose levels of 0 (vehicle), 1, 10, or 100 mg/kg/dose for 4 weekly doses. Reversibility of toxicity was evaluated during a 7-week recovery period following the final administration.

HCD80ECD:hIgG1Fc was clinically well tolerated in rats up to 100 mg/kg. At the 100 mg/kg dose, changes in hematologic parameters were observed, including increases in neutrophils, lymphocytes, and monocytes; a slight decrease in red blood cells (RBCs) and an increase in reticulocytes. Changes in clinical chemistry parameters were mostly seen at 100 mg/kg, including a decrease in triglycerides, an increase in alanine aminotransferase (ALT) and alkaline phosphatase (ALP), a decrease in albumin and an increase in globulins, with an associated decrease in the albumin/globulin ratio. Microscopic changes were observed in male and female rats at doses of 10 and 100 mg/kg, including mononuclear cell inflammation in multiple tissues, changes in lymphoid tissue, hepatic changes, and mononuclear cell infiltrates in the thyroid gland and kidney. Mononuclear cell inflammation was seen in the stomach, intestine, pancreas, salivary gland, and Harderian gland and was primarily observed at 100 mg/kg with only rare and minimal findings at 10 mg/kg. Increased lymphoid cellularity was observed in lymph nodes, spleen, and gut-associated lymphoid tissue (GALT) and was also primarily observed at 100 mg/kg, with lower frequency and less extensive changes observed at 10 mg/kg. Hepatic changes observed at 100 mg/kg included increased cellularity, hepatocellular hypertrophy, extramedullary hematopoiesis, mononuclear cell infiltrates, lymphoid/histiocytic aggregates, and necrosis with mixed cell infiltrates. In conclusion, the no-observed-adverse-effect level (NOAEL) in the pivotal rat study was determined to be 10 mg/kg for 4 weekly doses due to the treatment-related effects of the more severe mononuclear cell inflammation in the pancreas, gastrointestinal tract, salivary, and Harderian glands observed at 100 mg/kg.

In the pilot repeat-dose toxicology study, cynomolgus monkeys received 4 weekly IV doses of 0 (vehicle), 1, 10, and 50 mg/kg of hCD80ECD:hIgG1Fc. All dose levels were well tolerated by cynomolgus monkeys. Immunophenotyping analysis showed hCD80ECD:hIgG1Fc-related dose-dependent expansion and proliferation of central memory T-cells in the 10 mg/kg and 50 mg/kg dose groups, but not in the 1 mg/kg group. Histopathologically, at terminal necropsy, increased numbers of mononuclear cell infiltrates were seen in the liver, follicular hypertrophy was seen in the spleen and mesenteric lymph node, and increased cellularity of the bone marrow was seen at all dose levels. These findings resolved following the 6-week recovery period.

In the repeat-dose GLP toxicology studies in cynomolgus monkeys, hCD80ECD:hIgG1Fc protein was administered at dose levels of 0 (vehicle), 1, 10, or 100 mg/kg/dose for 4 weekly doses. Reversibility of toxicity was evaluated during a 6-week recovery period following administration of the last dose.

HCD80ECD:hIgG1Fc was well tolerated and no clinical or pathological changes were identified at 1 mg/kg when given as 4 weekly doses, but hCD80ECD:hIgG1Fc was not tolerated at doses of 10 and 100 mg/kg, necessitating unscheduled sacrifice and necropsy of 6/10 and 4/10 animals, respectively, between study days 14 and 30.

The affected animals displayed weight loss and lethargy, had signs consistent with dehydration, and were cold to the touch. Some monkeys had sporadic diarrhea. Significant body weight loss was observed several days prior to euthanasia. Affected animals showed significant electrolyte imbalance, including hyponatremia, blood urea nitrogen (BUN) and creatinine elevation, and signs of acute phase reaction (increased fibrinogen, increased globulin, increased C-reactive protein [CRP], and decreased albumin). Aldosterone and cortisol level were increased and adrenocorticotropic hormone (ACTH) decreased. Hematologic analysis showed a severe reduction of reticulocytes in 5 animals. No coagulation changes were observed. Serum cytokine measurements (IL-1, IL-2, IL-4, IL-6, IL-8, IL-10, IFN-γ, TNF-α, and granulocyte-macrophage colony-stimulating factor [GM-CSF]) on the day of unscheduled euthanasia showed signs of acute stress responses (TNF-α and IL8 increases), but the pattern of affected cytokines as well as the magnitude of changes did not indicate an acute cytokine release syndrome (CRS), i.e., no increase in IL2 or IL6.

Treatment-related pathological findings in the unscheduled necropsy animals were predominantly seen in large intestine and lymphoid tissues, with possible treatment-related microscopic changes in the kidneys and adrenals. In the digestive tract, mucosal erosion, crypt dilatation, and/or infiltration of mononuclear cells in the lamina propria of the large intestine, specifically the rectum, were observed. The observed changes in the lymphoid system include changes in the lymphoid cellularity (increases and decreases) of the inguinal, mandibular, and mesenteric lymph nodes. Decreased lymphoid cellularity was observed in the spleen and thymus. Findings of uncertain relationship to hCD80ECD:hIgG1Fc included an increased incidence of tubular dilatation with tubular casts and mineralization in the kidney, and an increased incidence of adrenal hypertrophy (zona fasciculata) in the adrenal.

In the surviving animals in the 10 mg/kg and 100 mg/kg group, the clinical observations of reduced body weight and decreased activity that were common with unscheduled euthanasia animals were also seen in 2 animals that reached scheduled euthanasia. Sporadic minimal to mild diarrhea was seen with higher incidence in animals administered 10 mg/kg and 100 mg/kg. HCD80ECD:hIgG1Fc-related changes in clinical chemistry parameters in the 10 mg/kg and 100 mg/kg group included a mild reduction in albumin and a mild increase in globulin at 10 mg/kg and 100 mg/kg. These changes were accompanied by increased fibrinogen, suggestive of an acute phase response. These changes returned to baseline at the end of the recovery period. These changes in clinical chemistry were not observed in the animals that survived to scheduled necropsy. No signs indicative of CRS, such as fever or cytokine increases consistent with CRS events, were observed.

Ophthalmic examination and cardiac evaluation did not show any hCD80ECD:hIgG1Fc-related changes at any dose level. At the scheduled necropsy, histopathological mucosal erosion and crypt dilatation were seen in the large intestine of animals given 100 mg/kg with sporadic findings in animals given 10 mg/kg. Also, at the scheduled necropsy, increased lymphoid cellularity was observed in the lymph nodes, whereas decreased lymphoid cellularity was observed in the spleen and thymus.

Overall, the histopathological changes were not of a magnitude that would explain the observed moribundity at doses of ≥10 mg/kg. The changes observed in the intestine were minimal to mild, and the diarrhea was sporadic among the affected animals. The timing and magnitude of changes in cytokine levels were not consistent with acute CRS and were more consistent with a stress response. Hyponatremia combined with the elevated BUN and creatinine could be indicative of renal or adrenal/pituitary effects; however, the histopathological findings in the kidney and adrenal were minimal and no histopathological findings were detected in the pituitary gland. The observed dehydration could be indicative of primary renal toxicity, however only minimal histopathological kidney damage was identified, and the lack of urinalysis at the time of euthanasia limits interpretation. Changes in ACTH, aldosterone, and cortisol hormone levels could indicate underlying endocrinopathy, however, these changes could also be explained by fluid loss and a compensatory stress response.

In summary, hCD80ECD:hIgG1Fc was clinically well tolerated in rat, and the NOAEL in rats is considered 10 mg/kg for 4-weekly doses. In cynomolgus monkeys, based on the GLP-toxicology study, doses of 10 mg/kg and 100 mg/kg were not tolerated. Some monkeys at the 10 mg/kg dose had sporadic diarrhea, dehydration, lethargy, and were cold to the touch. Intravenous hydration only temporarily improved the symptoms. Diffuse lymphocytic and monocytic infiltrates were observed in a variety of organs, however, the mechanism of this toxicity is undetermined. No clinical observations or adverse findings were seen in the low dose group of 1 mg/kg, which was, therefore, determined to be the NOAEL. The starting dose of 0.07 mg (0.001 mg/kg for a 70 kg human) has been calculated based on the minimum anticipated biologic effect level (MABEL) approach (see Example 7 below) and is approximately 1000-fold below the NOAEL. Significant anti-tumor activity is evident even at doses as low as 0.1 mg/kg in the CT26 tumor model, which is approximately 10-fold below the NOAEL in both rats and monkeys. Therefore, a potential therapeutic window for hCD80ECD:hIgG1Fc exists.

### Example 7: Selection of Initial Dose for Human Patients

A conservative starting dose based on the MABEL approach, close patient monitoring, staggered enrollment, and cautious dose escalation was designed to limit the risk to patients.

The MABEL approach was used because hCD80ECD:hIgG1Fc functions through two key T-cell regulators or modulators, including co-stimulation of CD28 on T-cells after T-cell receptor engagement, and blocking of CTLA-4 from competing for endogenous CD80. For hCD80ECD:hIgG1Fc, assessments of receptor occupancy (RO) and pharmacological activity (PA) through both CTLA-4 and CD28 were considered. To project the human dose based on Cₘₐₓ, assumptions of a plasma volume of distribution of central compartment of 2800 mL and a 70 kg average patient weight were used in calculating the percent RO and PA.

Integrating the assessments of RO and PA through both CTLA-4 and CD28, a starting dose of 0.07 mg was selected. Among the PA assays examined, CTLA-4 ELISA was thought to be both biologically relevant and sensitive. Using this ELISA assay, 50% PA leads to a predicted starting dose, when rounded down, of 0.07 mg. Several PA assays for CD28 activity were considered. However these assays were either thought to be not biologically relevant or predicted a much higher starting dose.

A Q3W dosing interval was selected. Although the half-life of hCD80ECD:hIgG1Fc in human patients is predicted to be less than 10 days, preclinical evidence suggests that the total exposure, not C_{trough}, may be an important driver of efficacy. The starting dose of 0.07 mg is predicted to attain a nominal (<1%) PA for CD28 using the binding assay of Chinese hamster ovary (CHO) cells overexpressing CD28. The dose escalation cohorts, along with the predicted PA for CD28 and CTLA-4 at each dose level at Cₘₐₓ, is summarized below (Table 2). During dose escalation, hCD80ECD:hIgG1Fc is projected to achieve 99% PA for CTLA-4 at Cₘₐₓ for doses ≥7 mg. Based on the K_{D} and observed Cₘₐₓ, ipilimumab, an anti-CTLA4 antibody, was projected to achieve 99% RO for CTLA-4 at the clinically approved dose of 3 mg/kg.

TABLE 2
hCD80ECD:hIgG1Fc Dose Selection

| Cohort | Dose Level (mg) | CD28 PA (%)* | CTLA-4 PA (%)** |
|---|---|---|---|
| 1aM1 | 0.07 | 0.16 | 42 |
| 1aM2 | 0.21 | 0.47 | 69 |
| 1aM3 | 0.70 | 1.5 | 88 |
| 1aM4 | 2.1 | 4.5 | 96 |
| 1aM5 | 7.0 | 14 | 99 |
| 1aM6 | 21.0 | 32 | >99 |
| 1aM7 | 42.0 | 48 | >99 |
| 1aM8 | 70.0 | 61 | >99 |

*PA estimation based on IC₅₀ of 16,000 ng/ml from cell binding assay using CD28 overexpressed CHO cell lines.
**PA estimation based on EC₅₀ of 34 ng/mL from hCD80ECD:hIgG1Fc CTLA-4 binding ELISA

Thus, the selected human doses take into account RO and PA through both CD28 and CTLA-4. Fixed 3-fold escalation increments are proposed while PA of CD28 is low, with more conservative increments (2-fold or less) proposed at higher expected CD28 activity levels.

### Example 8: Phase 1a Dose Escalation and Exploration Study

A phase 1a open-label multicenter study is conducted in up to 78 patients with advanced solid tumors using hCD80ECD:hIgG1Fc. Some patients may be enrolled at one or more dose levels. The patients in this study have advanced solid tumors, except central nervous system tumors. The patients are refractory to all standard therapies for their malignancy or are patients for whom standard therapies would not be appropriate.

### (A) Study Design

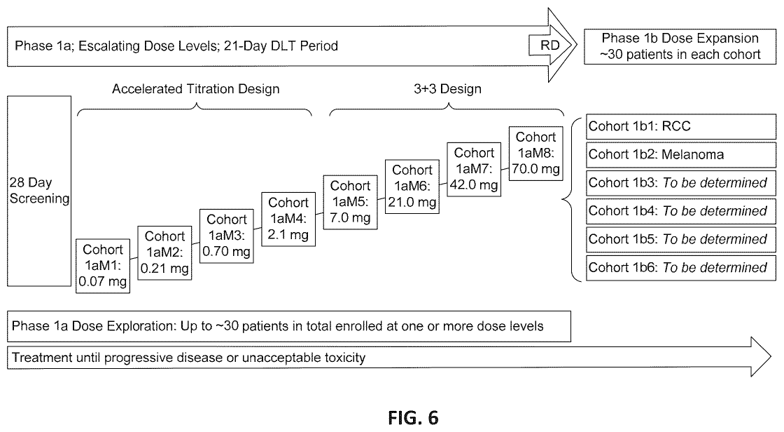

Phase 1a includes a Dose Escalation phase and a Dose Exploration phase. The Phase 1a study schema is provided in FIG. 6. In both the Dose Escalation and Dose Exploration phases, hCD80ECD:hIgG1Fc is administered as a 60-minute intravenous (IV) infusion every three weeks (Q3W) on Day 1 of each 21-day cycle. HCD80ECD:hIgG1Fc is administered as a flat dose.

The Phase 1a Dose Escalation includes an initial accelerated titration design followed by a standard 3+3 dose escalation design until the recommended dose (RD) for Phase 1b is determined. Up to 48 patients participate in the Dose Escalation phase. Doses from 0.07 mg to 70 mg are administered per the cohorts outlined in Table 3 below, and patients' second doses are at least 21 days after their first doses.

As immuno-oncology agents are associated with delayed immune-mediated toxicities, toxicities observed both during and beyond the 21-day dose-limiting toxicity (DLT) evaluation period are evaluated.

TABLE 3
Dose Levels for Accelerated Titration Design and 3 + 3 Design

| | | Dose of the | | |
|---|---|---|---|---|
| Design | Cohort | hCD80ECD:hIgG1Fc | | Regimen |
| Accelerated titration design | 1aM1 | 0.07 | mg | Q3W |
| | 1aM2 | 0.21 | mg | Q3W |
| | 1aM3 | 0.70 | mg | Q3W |
| | 1aM4 | 2.1 | mg | Q3W |
| 3 + 3 design | 1aM5 | 7.0 | mg | Q3W |
| | 1aM6 | 21.0 | mg | Q3W |
| | 1aM7 | 42.0 | mg | Q3W |
| | 1aM8 | 70.0 | mg | Q3W |

During Phase 1a Dose Escalation, the Dose-Limiting Toxicity (DLT) evaluation begins on the first day of treatment upon start of infusion and continues for 21 days. A DLT is defined as any of the following as related hCD80ECD:hIgG1Fc: (i) Absolute Neutrophil Count (ANC) is less than 1.0×10⁹ per L for more than 5 days or Grade 3 febrile neutropenia (e.g., ANC less than 1.0×10⁹ per L with a single temperature of more than 38.3° C. or fever more than 38° C. for more than 1 hour); (ii) platelets are less than 25×10⁹ per L or platelets are less than 50×10⁹ per L with clinically significant hemorrhage; (iii) aspartate aminotransferase/alanine transaminase (AST/ALT) is more than 3 times the upper limit of normal (ULN), and concurrent total bilirubin is more than twice ULN not related to liver involvement with cancer; (iv) Grade 3 or higher non-hematologic toxicity (except Grade 3 fatigue lasting less than 7 days; Grade 3 nausea and Grade 3-4 vomiting and diarrhea lasting less than 72 hours in patients who have not received optimal anti-emetic and/or anti-diarrheal therapy; Grade 3 endocrinopathy that is adequately treated by hormone replacement; and/or laboratory value that may be corrected through replacement within 48 hours); and/or (v) Grade 2 neurological toxicity except headache and peripheral neuropathy in patients with Grade 1-2 peripheral neuropathy at entry.

An accelerated titration design enrolling at least 1 patient at each dose level is carried out for dose levels 0.07, 0.21, 0.7 and 2.1 mg. Dose escalation to the next dose level proceeds after at least 1 patient completes the 21-day DLT evaluation interval. If a single patient experiences a DLT during the 21-day evaluation interval, standard 3+3 dose escalation criteria applies for that cohort as well as all subsequent dosing cohorts. If at least 2 patients experience moderate adverse events (AE) (at any accelerated titration dose level), standard 3+3 dose escalation criteria will apply for the highest dose level at which a moderate AE was experienced, with enrollment of additional patients. All subsequent dosing cohorts will then follow the standard 3+3 dose escalation criteria. Moderate AEs are defined as ≥Grade 2 AEs as related to hCD80ECD:hIgG1Fc. Grade 2 laboratory values are not considered as moderate AEs for this purpose unless accompanied by clinical sequelae.

Intra-patient dose escalation will be permitted in patients enrolled at dose levels below 7.0 mg provided: (i) the patient did not experience a DLT; (ii) all other AEs have recovered to Grade 1 or lower prior to dose escalation; (iii) the patient may only dose escalate by a maximum of 1 dose level every 21 days and only after that dose level has cleared DLT review; and (iv) the patient cannot dose escalate beyond the 7.0 mg dose level.

The algorithm outlined in Table 4 below is used for all standard 3+3 dose escalations.

TABLE 4
Phase 1a Algorithm for 3 + 3 Dose Escalation Decisions

| Number of Patients with | |
|---|---|
| DLT at a Given Dose Level | Dose Escalation Decision Rule |
| 0/3 | Enroll 3 patients at next dose level (next/higher cohort) |
| 1/3 | Enroll 3 additional patients at current dose level (current cohort) |
| 2/3 | Stop enrollment. If at Cohort 1aM1, the study will be stopped. If at |
| | Cohort 1aM2 or above, enroll 3 more patients at the previous dose level |
| | (previous/lower cohort) if only 3 were previously enrolled, or at an |
| | intermediate dose level |
| 1/6 | Enroll 3 patients at next dose level (next/higher cohort) |
| ≥2/6 | Stop enrollment. If at Cohort 1aM1, the study will be stopped. If at |
| | Cohort 1aM2 or above, enroll 3 more patients at the previous dose level |
| | (previous/lower cohort) if only 3 were previously enrolled, or at an |
| | intermediate dose level |

The maximum tolerated dose (MTD) and/or recommended dose (RD) of hCD80ECD:hIgG1Fc for Phase 1a is identified based on an evaluation of the overall safety, tolerability, pharmacodynamics, pharmacokinetics, and preliminary efficacy. The MTD will be a dose level where no more than 1/6 patients report a DLT. The RD will be identified based on an evaluation of all available safety, tolerability, pharmacokinetic, and pharmacodynamics data. The RD will consider toxicities observed both during and beyond the DLT evaluation period as well as dose reductions and discontinuations due to toxicity that do not meet the DLT criteria. The RD, therefore, may or may not be the same as the identified MTD. For example, if the MTD is not reached, or if data from subsequent cycles of treatment from Phase 1a provide additional insight on the safety profile, then the RD may be a different, though not higher, dose than the MTD.

The Phase 1a Dose Exploration cohort enrolls up to 30 patients in total who may be enrolled at one or more dose levels to further evaluate safety, pharmacokinetics, pharmacodynamics, and clinical activity. Toxicities observed in these patients will contribute to the overall assessments of safety and tolerability, and may inform selection of the RD. Clinical activity may be evaluated in specific tumor types based on safety, pharmacokinetic, pharmacodynamic, and efficacy data.

Cytokine levels, including circulating TL-6, TNF, and IFNγ levels are monitored.

### (B) Subjects

A total of up to 78 patients in Phase 1a are identified based on the following inclusion and exclusion criteria.

Patients in Phase 1a meet all of the following inclusion criteria:
Patients must be 18 years of age or older
Histologically confirmed solid tumors (except primary central nervous system tumors);
Disease that is unresectable, locally advanced, or metastatic and has progressed following all standard treatments (i.e., refractory) or is not appropriate for standard treatments;
At least one measurable lesion at baseline according to RECIST v1.1; tumor sites situated in a previously irradiated area, or in an area subjected to other loco-regional therapy, are not considered measurable unless there has been demonstrated progression in the lesion;

Patients in Phase 1a are excluded from the study if any of the following criteria apply:
Treatment with any anti-cancer therapy or participation in another investigational drug or biologics trial within 28 days or ≤5 half-lives (whichever is shorter) prior to first dose of study treatment administration or while on this study;
For patients participating in Phase 1a dose escalation and exploration cohorts: Prior treatment with a CTLA-4 antagonist, including ipilimumab and tremelimumab;
Patients who have received prior immune-modulating therapies (including regimens containing an immune agonist or a programmed death-ligand 1 ([PD-L1]/programmed cell death protein 1 [PD-1] antagonist) are NOT permitted to enroll unless all the following apply: (a) must not have experienced a drug-related toxicity that led to permanent discontinuation of prior immunotherapy and (b) treatment was administered 5 half-lives or 90 days (whichever is shorter) prior to first dose of study treatment;
Ongoing adverse effects from prior treatment >National Cancer Institute Common Terminology Criteria for Adverse Events (NCI CTCAE) Grade 1 (with the exception of Grade 2 alopecia or peripheral neuropathy);
Severe allergic, anaphylactic, or other infusion-related reaction to a previous biologic agent;

### (C) Results

The incidence of AEs, serious AEs, clinical laboratory abnormalities, and electrocardiogram (ECG) abnormalities are evaluated to show that hCD80ECD:hIgG1Fc is safe and tolerable in patients with advanced solid tumors. The incidence of AEs defined as dose-limiting toxicities, clinical laboratory abnormalities defined as dose-limiting toxicities, and overall assessment of pharmacokinetics and pharmacodynamics are evaluated to determine the recommended dose of hCD80ECD:hIgG1Fc.

Pharmacokinetic parameters (AUC, Cₘₐₓ, C_{trough}, CL, t_{1/2}, vₛₛ (volume of distribution at a steady state)) in patients with advanced solid tumors are determined from serum concentration-time data of hCD80ECD:hIgG1Fc using a non-compartmental analysis. Other parameters, such as dose proportionality, accumulation ratio, and attainment of steady state, will also be calculated if the data are available. Serum concentrations of hCD80ECD:hIgG1Fc are determined using the enzyme-linked immunosorbent assay (ELISA) method.

The impact of immunogenicity (i.e., anti-drug antibody immune responses to hCD80ECD:hIgG1Fc) in patients with advanced solid tumors on exposure to hCD80ECD:hIgG1Fc is assessed by measuring total antibodies against hCD80ECD:hIgG1Fc from all patients.

The clinical benefits of hCD80ECD:hIgG1Fc in human patients with advanced solid tumors are demonstrated. Tumor assessments include a clinical examination and imaging (e.g., computed tomography (CT) scans with appropriate slice thickness per RECIST v1.1 or magnetic resonance imaging (MRI)). Tumors are assessed at screening, every 6 weeks from the first dose for 24 weeks, then every 12 weeks thereafter to show inhibition of tumor growth and tumor regression (e.g., complete tumor regression). Once an initial CR or PR is noted, confirmatory scans must be performed 4 to 6 weeks later. A lack of significant increase in circulating IL-6, TNF, and IFNγ indicates that hCD80ECD:hIgG1Fc does not cause a cytokine storm.

The objective response rate (ORR) is also determined as a measure of efficacy. The ORR is defined as the total number of patients with confirmed responses (either complete response (CR) or partial response (PR) per RECIST v.1.1) divided by the total number of patients who are evaluable for a response.

After seven patients were treated with hCD80ECD:hIgG1Fc (doses ranging from 0.07-7 mg), no dose-limiting toxicities were observed. The median age of the seven patients was 58 years, and 57% of the patients had Eastern Cooperative Oncology Group Performance Status (ECOG PS) of 1. The median number of prior therapies was 4 (range: 2-8). Only two treatment-emergent adverse events (TEAEs) of Common Terminology Criteria for Adverse Events (CTCAE) grade 3 or higher were reported (bile duct obstruction, and new central nervous lesion; from disease progression in both cases). There were no serious adverse events, or ≥grade 3 TEAEs attributed to hCD80ECD:hIgG1Fc, and the only TEAE attributed to hCD80ECD:hIgG1Fc in more than one patient was fatigue (n=2).

### Example 9: Phase 1b Dose Expansion

A Phase 1b open-label multicenter study is conducted using hCD80ECD:hIgG1Fc in up to 180 patients with advanced solid tumors.

### (A) Study Design

Phase 1b is the dose expansion portion of the study. The Phase 1b study schema is provided in FIG. 6. Enrollment into Phase 1b Dose Expansion begins after identification of the maximum tolerated dose (MTD) and/or recommended dose (RD) in Phase 1a.

Phase 1b includes tumor-specific cohorts of up to 30 patients each as shown in Table 5. Patients with renal cell carcinoma or melanoma who have failed prior anti-PD(L)1 therapy are enrolled. Additional tumor types for the remaining four Phase 1b cohorts will be determined based on safety, translational, and safety information from other immunotherapies and changes to prescribing information for approved immunotherapies.

TABLE 5
Phase 1b Expansion Cohorts and Tumor Types

| Cohort | Tumor Type |
|---|---|
| 1b1 | Renal Cell Carcinoma |
| 1b2 | Melanoma |
| 1b3 | To Be Determined |
| 1b4 | To Be Determined |
| 1b5 | To Be Determined |
| 1b6 | To Be Determined |

HCD80ECD:hIgG1Fc is administered as a 60-minute intravenous (IV) dose every three weeks (Q3W) on Day 1 of each 21-day cycle. HCD80ECD:hIgG1Fc is administered as a flat dose.

### (B) Subjects

Up to 30 patients are enrolled into each specific Phase 1b cohort.

Patients in Phase 1b meet all of the following inclusion criteria:
All inclusion criteria for Phase 1a;
For Cohort 1b1—renal cell carcinoma
Histologically or cytologically confirmed advanced or metastatic renal cell carcinoma with a clear-cell component;
Patients must have received at least one prior anti-angiogenic therapy regimen (e.g., sunitinib, sorafenib, pazopanib, axitinib, tivozanib, or bevacizumab) in the advanced or metastatic setting; and
Patients must have received at least one anti-PD(L)1 therapy (e.g., nivolumab, pembrolizumab, atezolizumab, durvalumab, or avelumab) in the advanced or metastatic setting. Prior cytokine therapy (e.g., IL-2 or IFN-α) and anti-CTLA4 therapy (e.g., ipilimumab) is allowed, but not required.
For Cohort 1b2—melanoma
Patients with histologically or cytologically confirmed unresectable stage III or stage IV cutaneous melanoma, which is not amendable to local therapy;
Patients must have received at least one anti-PD(L)1 therapy (e.g., nivolumab, pembrolizumab, atezolizumab, durvalumab, or avelumab) in the advanced or metastatic setting. Prior cytokine therapy (e.g., IL-2 or IFN-α) and anti-CTLA4 therapy (e.g., ipilimumab) is allowed, but not required; and
Patients with BRAF mutations must have received prior BRAF inhibitor therapy (e.g., vemurafenib or dabrafenib) in the advanced or metastatic setting.

Patients must comply with the same exclusion criteria for Phase 1a to be included in the Phase 1b study.

### (C) Results

The incidence of AEs, serious AEs, clinical laboratory abnormalities, and electrocardiogram (ECG) abnormalities are evaluated to show that hCD80ECD:hIgG1Fc is safe and tolerable in patients with advanced solid tumors. The incidence of AEs defined as dose-limiting toxicities, clinical laboratory abnormalities defined as dose-limiting toxicities, and overall assessment of pharmacokinetics and pharmacodynamics are evaluated to determine the recommended dose of hCD80ECD:hIgG1Fc.

Pharmacokinetic parameters (AUC, Cₘₐₓ, C_{trough}, CL, t_{1/2}, vₛₛ (volume of distribution at a steady state)) in patients with advanced solid tumors are determined from hCD80ECD:hIgG1Fc serum concentration-time data using a non-compartmental analysis. Other parameters, such as dose proportionality, accumulation ratio, attainment of steady state, will also be calculated if the data are available. Serum concentrations of hCD80ECD:hIgG1Fc are determined using the enzyme-linked immunosorbent assay (ELISA) method.

The impact of immunogenicity (i.e., anti-drug antibody immune responses to hCD80ECD:hIgG1Fc) in patients with advanced solid tumors on exposure to hCD80ECD:hIgG1Fc is assessed by measuring total antibodies against hCD80ECD:hIgG1Fc from all patients.

The clinical benefits of hCD80ECD:hIgG1Fc in patients with advanced solid tumors are demonstrated. Tumor assessments include a clinical examination and imaging (e.g., computed tomography (CT) scans with appropriate slice thickness per RECIST v1.1 or magnetic resonance imaging (MRI)). Tumors are assessed at screening, every 6 weeks from the first dose for 24 weeks, then every 12 weeks thereafter to show inhibition of tumor growth and tumor regression (e.g., complete tumor regression). Once an initial CR or PR is noted, confirmatory scans must be performed 4 to 6 weeks later.

The objective response rate (ORR), duration of response (DOR), progression-free survival (PFS), disease control rate (DCR), and overall survival (OS) are also determined as a measure of efficacy. The ORR is defined as the total number of patients with confirmed responses (either complete response (CR) or partial response (PR) per RECIST v.1.1) divided by the total number of patients who are evaluable for a response. The DOR is defined as the time from first response (CR or PR per RECIST v1.1) that is subsequently confirmed until the onset of progressive disease or death from any cause, whichever comes first. PFS is defined as the time from the patient's first dose to the first observation of disease progression or death due to any cause, whichever comes first. DCR is defined as the total number of patients with confirmed responses of either CR, PR, or stable disease as per RECIST v1.1 divided by the total number of patients who are evaluable for a response. OS is defined as the time from the first dose of hCD80ECD:hgGFc until death from any cause.

### Example 10: Selection of Higher HCD8rECD:hIgG1Fc Dose

HCD80ECD:hIgGrFc was administered to human patients per the protocols described in Examples 8 and 9. The characteristics of the treated patients are summarized in Table 6.

TABLE 6
Characteristics of Patients Treated with HCD80ECD:hIgG1Fc

| Dose (mg) | 0.07 | 0.21 | 0.7 | | 2.1 | 7 | | 21 | | 42 | |
|---|---|---|---|---|---|---|---|---|---|---|---|
| Number of patients | 1 | 1 | 2* | | 1 | 4\hat{ } | | 3 | | 3 | |
| Median Age (Range) | 53 | 61 | 66 | (58, 73) | 42 | 66 | (54-73) | 63 | (60-70) | 68 | (65-74) |
| Number of prior | 4 | 2 | 5.5 | (4-7) | 6 | 5 | (3-7) | 5 | (3-5) | 2 | (2-3) |
| cancer regimens | | | | | | | | | | | |

| | | | | | | | |
|---|---|---|---|---|---|---|---|
| Type(s) of cancer | Colorectal | Mesothelioma | Cervical | Colorectal | Cholangio | Colorectal | Gallbladder |
| | | | Breast | | Colorectal | Colorectal | Mesothelioma |
| | | | | | Lung Pancreas | Lung | Thyroid |
| Median duration of | 41 | 190 | 64 | 63 | 57 | 42 | 42 |
| exposure (days) | | | | | | | |

*Two patients identified for participation simultaneously (not due to adverse events)
\hat{ }1 patient in dose-expansion

In these patients, no dose limiting toxicities or adverse events of grade 4 or higher were observed. There were five serious adverse events (including deep vein thrombosis, bile duct obstruction, new CNS lesion; recurrent pleural effusion, and herpes zoster), all due to underlying disease. Anti-drug antibodies were observed in 2 out 15 patients. No consistent treatment emergent elevation in cytokines was observed, and no dose-response between hCD80ECD:hIgG1Fc and cytokine elevation was observed. In addition, no significant treatment-related changes in peripheral T cell compartment were observed.

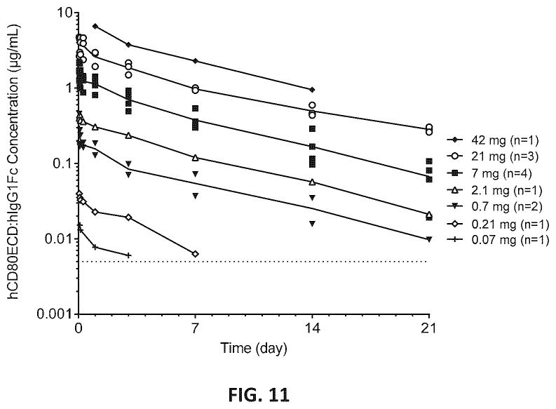

The serum concentrations of hCD80ECD:hIgG1Fc in patients following the first dose are shown in FIG. 11. Linear clearance was observed following doses from 7 mg to 21 mg. the elimination half-life was approximately 1 week after the single dose. Minimal accumulation was observed following repeat dosing every three weeks (Q3W).

Stable disease occurred as follows: 0/1 patient receiving 0.7 mg, 1/1 patient receiving 0.21 mg, 1/2 patients receiving 0.7 mg, 1/1 patient receiving 2.1 mg, 1/4 patients receiving 7 mg, and 0/3 patients receiving 21 mg. Conversely, progressive disease occurred as follows: 1/1 patient receiving 0.7 mg, 0/1 patient receiving 0.21 mg, 1/2 patients receiving 0.7 mg, 0/1 patient receiving 2.1 mg, 3/4 patients receiving 7 mg, and 3/3 patients receiving 21 mg.

The percentages of CTLA-4 receptor occupancy associated with the Cₘₐₓ and C_{trough} values observed in patients receiving 0.07 mg to 21 mg were calculated based on the binding affinity of hCD80ECD:hIgG1Fc for CTLA-4 (using surface plasmon resonance at 1.8 nM). The Cₘₐₓ and C_{trough} values for higher doses were projected (assuming linear clearance), and the CTLA-4 receptor occupancy for these doses were also calculated. The results are shown in Table 7.

TABLE 7
CTAL-4 Receptor Occupancy for HCD80ECD:hIgG1Fc Doses

| Dose | Cₘₐₓ | | C_{trough} | |
|---|---|---|---|---|
| (mg) | (μg/mL) | RO % at Cₘₐₓ | (μg/mL) | RO % at C_{trough} |
| 0.07 | 0.0152 | 7.80 | NA | NA |
| 0.21 | 0.190 | 51.3 | NA | NA |
| 0.7 | 0.288 | 61.6 | 0.00974 | 5.14 |
| 2.1 | 0.444 | 71.2 | 0.0211 | 10.5 |
| 7 | 1.67 | 90.3 | 0.0564 | 23.9 |
| 21 | 4.05 | 95.8 | 0.282 | 61.1 |
| 42 | 8.28 | 97.9 | 0.566 | 75.9 |
| 70 | 13.8 | 98.7 | 0.943 | 84.0 |
| 140 | 27.6 | 99.4 | 1.89 | 91.3 |
| 280 | 55.2 | 99.7 | 3.77 | 95.5 |
| 560 | 110 | 99.8 | 7.55 | 97.7 |
| 700 | 138 | 99.9 | 9.43 | 98.1 |

Observed Cmax and Ctrough values are shown in italics with underline.
Other Cmax and Ctrough values are projected values based on pharmacokinetic dates from 3 patients at 21 mg doses (assuming linear clearance)

In order to achieve a desired receptor occupancy of about 60% to about 98.5%, doses of 140-700 mg were selected.

### Example 11: Gene Expression Analysis of Granzyme B and Interferon Gamma in Tumor-Bearing and Naïve BALB/c Mice Treated with Murine CD80 ECD-Fc

Immuno-competent BALB/c mice were inoculated with CT26, a murine colorectal carcinoma, and treatment with murine CD80 ECD-Fc was administered IV when tumors reached approximately 100 mm³. Murine CD80 ECD-Fc is a mouse surrogate fusion protein comprising the extracellular domain (ECD) of murine CD80 linked to the Fc domain of mouse IgG2a wild type (mCD80-Fc). Murine CD80 ECD-Fc was evaluated at four dose levels: 0.03 mg/kg, 0.1 mg/kg, 0.3 mg/kg, and 0.9 mg/kg. To assess gene expression changes in naïve animals, non-tumor-bearing BALB/c mice were administered 0.9 mg/kg, 10 mg/kg, or 50 mg/kg mCD80-Fc. As negative controls, mice were administered 0.9 mg/kg (tumor-bearing) or 50 mg/kg (naïve) mIgG2a isotype control. Samples were collected for transcriptomic analysis 11 days post-dose. Tumors were resected and snap-frozen in liquid nitrogen, and blood samples were collected in Qiagen RNAprotect animal blood tubes (100 μl). RNA was isolated and used to prepare targeted sequencing libraries (Mouse Immuno-Oncology kit, Qiagen RMM-009Z). Tumor libraries and blood libraries were run separately. Blood DNA libraries were sequenced at higher sequencing depth for increased sensitivity.

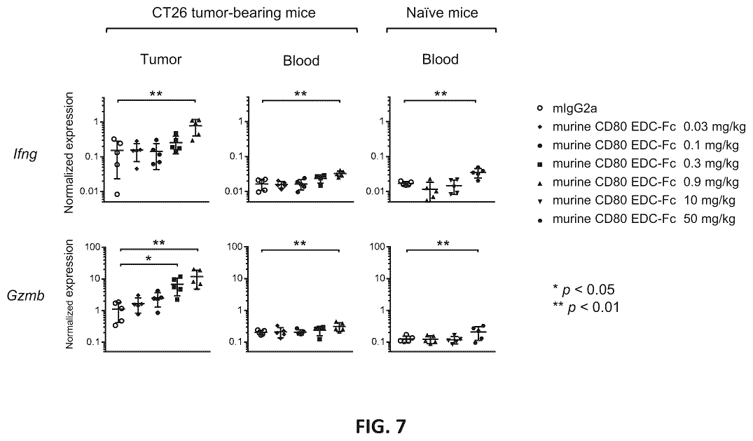

To determine dose dependency of changes in the tumor and blood, two markers of T cell activation were evaluated. The results are shown in FIG. 7. Granzyme B (Gzmb) showed a dose-dependent upregulation in the tumor, with significance reached at the two highest dose levels of mCD80-Fc, 0.3 mg/kg and 0.9 mg/kg. This upregulation was also observed in the blood of tumor-bearing animals at the same dose levels, with significance reached at 0.9 mg/kg mCD80-Fc. By contrast, mCD80-Fc treatment did not impact Gzmb expression in non-tumor-bearing animals except at as the highest dose level tested, 50 mg/kg. Interferon gamma (Ifng) was significantly upregulated at 0.9 mg/kg both in tumor and blood from tumor-bearing mice, with a small trend towards increased expression at 0.3 mg/kg in both compartments. Murine CD80 ECD-Fc treatment only upregulated Ifng expression in blood from naïve animals at 50 mg/kg. These data indicate that mCD80-Fc has preferential activity in the tumor microenvironment, and that non-specific polyclonal T cell activation is not observed at dose levels up to 10 mg/kg. These data also indicate that mCD80-Fc induces T cell activation in tumor-bearing animals at the proposed clinical dose levels. Taken together, the data suggest that hCD80ECD:hIgG1Fc would have specific activity in the tumor microenvironment of patients at the proposed clinical dose levels, further supporting both the safety and efficacy of this molecule.

### Example 12: CD80 ECD-Fc Activity in Whole Blood Mixed Lymphocyte Reactions

HCD80ECD:hIgG1Fc was tested in vitro in primary T cells assays using pooled, irradiated PBMC from multiple donors to stimulate individual donor blood T cells (Bromelow et al., Journal of Immunological Methods 247: 1-8 (2000). Alloreactive T cells are found at high frequencies in the blood and react to a variety of peptide:MHC presented on the surface of irradiated PBMC, which also express Fc receptor (FcR) that can bind hCD80ECD:hIgG1Fc and mediate co-stimulation of responding T cells. This format allows the testing of hCD80ECD:hIgG1Fc activity with physiologically-relevant antigen presenting cell (APC) populations, and the use of pooled PBMC helps to reduce donor to donor variability in T cell responses.

Human whole blood samples were processed for PBMC isolation from individual donors and irradiated with 5000-6000 rads. Then equal numbers of PBMCs from each donor were pooled at a final concentration of 1×10⁶ cells/mL in RPMI-10 (Roswell Park Memorial Institute 1640 medium supplemented with 2 mM L-glutamine, 25 mM Hepes, 1× Penicillin/Streptomycin, 2ME and 10% human serum.

Test conditions were prepared at 4× the desired final concentration in media, and the following were combined per well in a 96-well U-bottom tissue culture plate:
50, 25, or 12.5 μL of irradiated PBMC (8, 4, and 2×10⁵ PBMC, respectively) with RPMI-10 supplemented to 50 μL total;
50 μl of 1000, 500, or 250 μg/mL Fc-Hinge control or hCD80ECD:hIgG1Fc for final concentrations of 250, 125, and 62.5 μg/mL;
50 μl of media containing antibody: anti-CD32 at 40 μg/mL (final 10 μg/mL), anti-CD28 at 4 ug/mL (final 1 μg/mL), anti-PDL1 at 40 μg/mL (final 10 μg/mL), or Ipilimumab at 40 ug/mL (final 10 μg/mL);
50 μl of whole blood diluted in RPMI with no serum (30 μL RPMI with 20 μL whole blood, containing ˜2×10⁴ T cells)

Plates were incubated at 37° C. in 5% CO₂ for 5 days, supernatants were removed, and cells were resuspended in RPMI −10 containing 10 μM ethynl deoxyuridine (EdU). An aliquot of each condition was collected and incubated with anti-CD3 (OKT3, 10 μg/mL) and anti-CD28 (CD28.2, 2 μg/mL). Cells were incubated for an additional 24 hours, and anti-CD3/CD28-stimulated cells were cultured for 5 more hours following the addition of brefeldin A. Cells were then washed in PBS, centrifuged, and resuspended in 100 μL Live/Dead NearIR viability dye prepared and diluted in 1×PBS according to the manufacturer's instructions and then incubated for 20 minutes at 4° C. Cells were pelleted by centrifugation, and three separate staining panels for T cell phenotype and function were added to the samples in 100 μl of FACS buffer and then incubated for 30 minutes at 4° C. The cells were then labeled with FoxP3, intracellular cytokine staining, and Clik-iT EdU.

Samples were acquired on a BD LSRFortessa and analyzed using FlowJo, Excel, and Graphpad Prism software. Briefly, singlet events were identified by comparing scatter characteristics, and T cells were identified as Lineage-(CD14−, CD15−, CD19−, and CD56−), CD3+, CD4+ or CD8+ cells. In some experiments, cell-surface markers of activation were also assessed (e.g., CD25, CD95, PD1).

Secreted cytokines were measured in assay supernatants by colorimetric ELISA using a commercial kit according to the manufacturer's instructions. Assay plates were read using an Envision 2103, and the data were analyzed using Excel and Graphpad Prism Software

There were few cytokines produced when stimulated with 2×10⁵ or 8×10⁵ PBMC in the absence of costimulation. Furthermore, both CD4 and CD8 T cells showed little proliferation or activation-induced upregulation of CD25 without additional signaling. When cultures were supplemented with anti-CD28 antibody (clone 28.2), there was an increase in T cell activation in a PBMC stimulator-dependent matter. Low numbers of PBMC in conjunction with anti-CD28 only increased the expression of CD25 on CD4 T cells, while high numbers of PBMC increased IL-2 and IFN-γ secretion and stimulated proliferation and activation of both CD4 and CD8 T cells as measured by EdU incorporation and CD25 upregulation.

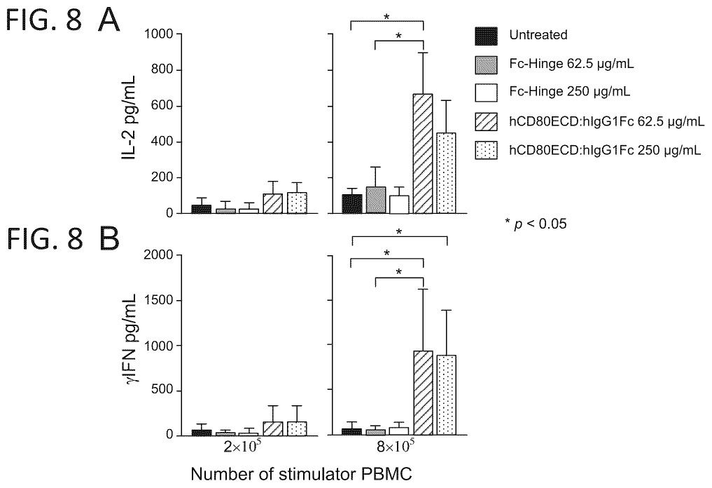

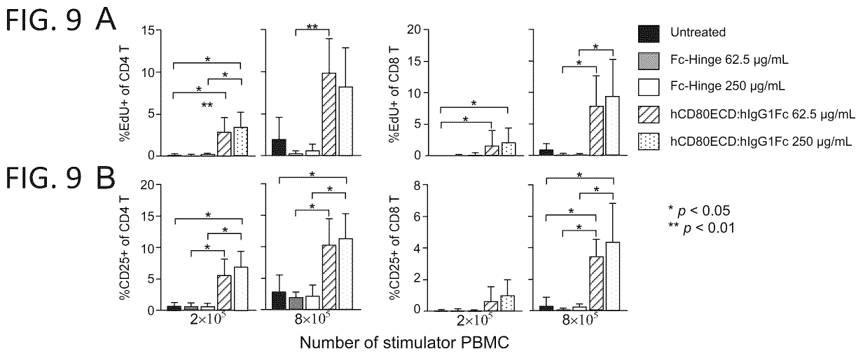

HCD80ECD:hIgG1Fc enhanced IL-2 and IFNγ secretion by T cells, and this effect was dependent upon the number of stimulator cells (FIG. 8). The maximal effect of hCD80ECD:hIgG1Fc was higher than that observed with saturating agonistic anti-CD28. HCD80ECD:hIgG1Fc and also increased the proliferation of CD4 and CD8 T cells and expression of CD25 in a stimulator-dependent manner (FIG. 9). However, unlike the cytokine levels, the increases in T cell proliferation were significant when stimulated with 2×10⁵ PBMC. CD4 T cell upregulation of CD25 was also observed following stimulation with low and high numbers of PBMC. HCD80ECD:hIgG1Fc did not activate T cells in the absence of TCR stimulation, as evidenced by control samples utilizing whole blood and autologous irradiated PBMC.

These assays demonstrated an enhancement of proliferation, cytokine, and activation marker responses by hCD80ECD:hIgG1Fc. The maximal responses were comparable to or higher than those observed with a saturating amount of a conventional anti-CD28 agonistic antibody. This costimulatory activity required the allogeneic TCR stimulus, indicating that hCD80ECD:hIgG1Fc did not have a TCR-independent superagonist activity.

Additional experiments assessed the complement activation of hCD80ECD:hIgG1Fc on primary human immune cells. One assay measured the binding of C1q to human immune cell-bound hCD80ECD:hIgG1Fc using PBMC left unactivated (expressing only CD80 ligands and CD28 and PD-L1) or activated to induce cell surface expression of CTLA-4 in addition to CD28 and PD-L1. Despite significant hCD80ECD:hIgG1Fc binding, no significant differences in C1q binding were detected between hCD80ECD:hIgG1Fc and hIgG1-Fc (control) treated cells indicating that C1q does not specifically engage hCD80ECD:hIgG1Fc when bound to primary human immune cells. Another assay measured CD4+ T cell lysis in the presence of hCD80ECD:hIgG1Fc and complement in vitro. Unactivated and activated CD4+ T cells were treated with hCD80ECD:hIgG1Fc and cultured in the presence of human serum complement. Cell lysis was measured, and hCD80ECD:hIgG1Fc did not result in CD4+ T cell death at any concentration tested. These results indicate that complement-dependent cytotoxicity CDC is not a mechanism of hCD80ECD:hIgG1Fc activity.

### Example 13: CD80 ECD-Fc is Active in 200 mm² Tumors

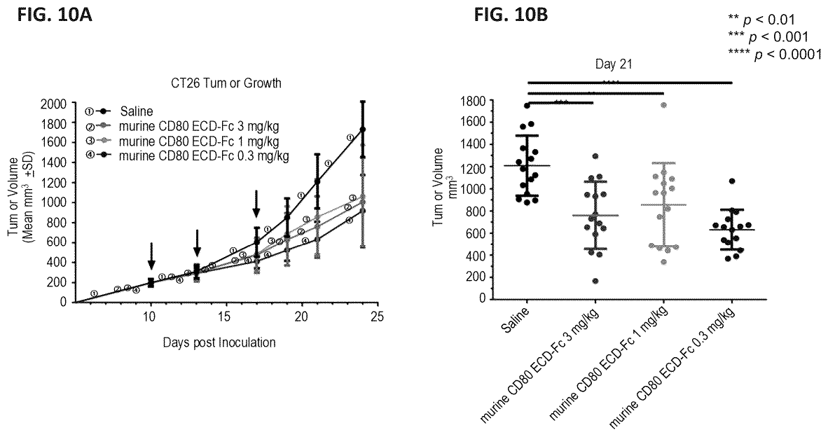

The activity of murine CD80 ECD-Fc on CT26 tumors is shown in above in Example 3. The activity of murine CD80 ECD-Fc on larger CT26 tumors was also evaluated. In these experiments, treatment was initiated on day 10, when tumor volumes had reached about 200 mm² (195-198 mm²). Specifically, on days 10, 13, and 17, mice (n=15 in each group) received saline, 0.3 mg/kg murine CD80 ECD-Fc, 1 mg/kg murine CD80 ECD-Fc, or 3 mg/kg murine CD80 ECD-Fc. As shown in FIG. 10, all three doses of murine CD80 ECD-Fc significantly inhibited the growth of CT26 tumors as compared to the saline treatment group.

### Example 14: Pharmacokinetic Analysis of CD80 ECD-Fc

An in vivo study was conducted in Balb/c mice to analyze the pharmacokinetics of six different lots of hCD80ECD:hIgG1Fc having different sialic acid (SA) contents.

CD80 ECD-Fc fusion protein was transiently expressed in CHO cells and cultured for 5-7 days. Clarified cell culture supernatant containing the secreted protein was purified by Protein A chromatography with a low pH elution step gradient. The Protein A purified pool was then adjusted to pH 8.0 and loaded onto strong anion exchanger chromatography column.

A linear salt gradient of (A) 20 mM Tris, 20 mM NaCl, pH 8.0 to (B) 20 mM Tris, 250 mM NaCl, pH 8.0 over twenty column volumes was applied to elute the bound protein. Two separate preparations were performed. The eluted peaks from each purification were divided into six separate pools (Lots provided Table 8). Each pool was buffer exchanged into 1×Dulbecco's phosphate-buffered saline (DPBS) by dialysis and analyzed for total sialic acid content analysis. Table 8 below shows the amount of sialic acid in each Lot.

TABLE 8
Amount of sialic acid in CD80 ECD-Fc
fusion protein in each fraction

| | Sialic Acid |
|---|---|
| Lot | (mol/mol) |
| Unfractionated | 14 |
| A | 5 |
| C | 10 |
| D | 15 |
| E | 20 |
| F | 26 |

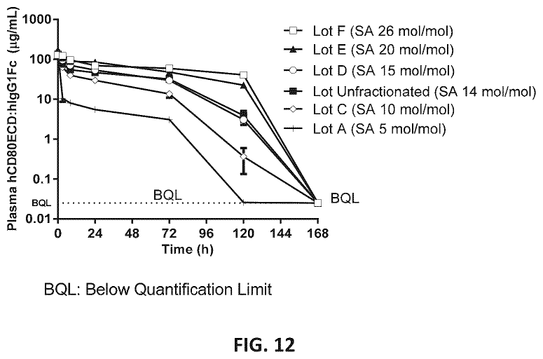

The mice were divided into six groups (2-5 mice per group) (from Table 8) and administered a single intravenous dose of 5 mg/kg CD80 ECD-Fc fusion protein. Serum concentrations of CD80 ECD-Fc fusion protein were determined using an enzyme-linked immunosorbent assay (ELISA) method at various times up to 168 hours after administration. FIG. 12 shows that the concentration of CD80 ECD-Fc fusion protein is higher in mice administered with CD80 ECD-Fc fusion protein higher amounts of sialic acid as compared to CD80 ECD-Fc fusion protein with lower amounts of sialic acid.

The amount of sialic acid on CD80 ECD-Fc fusion protein affects the distribution phase of the fusion protein following a single IV administration. As a result, the exposure of CD80 ECD-Fc fusion protein increases with increasing amounts of sialic acid until sialic acid is greater than or equal to 20 mol/mol, where the clearance of CD80 ECD-Fc fusion protein is saturated. The exposure between Lot D and Lot E are close enough such that similar efficacy was seen at the same dose. See FIG. 2. Thus, sialic acid content of at least 20 moles sialic acid per mole of fusion protein results in greater exposure than lower levels of sialic acid.

### Example 15: Pharmacokinetic and Safety of hCD80ECD:hIgG1Fc in Humans

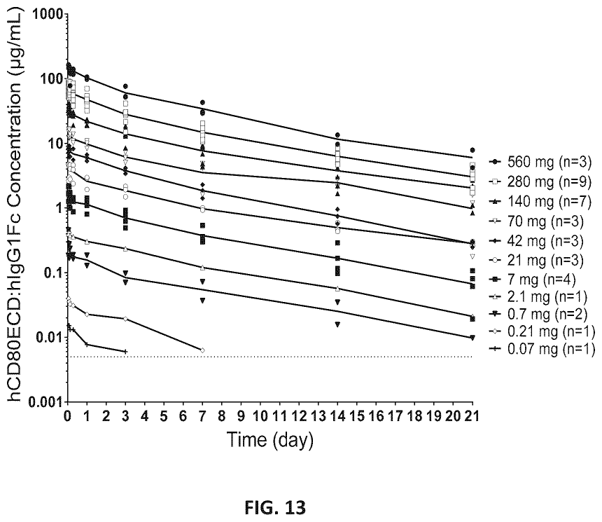

hCD80ECD:hIgG1Fc was administered as monotherapy in a dose range from 0.07 mg to 560 mg in 46 subjects with solid tumors in a Phase 1 study (generally as described in Examples 8, 9, and 10, but also including additional dose levels). Thirty-seven out of fourty-six patients had more than one measurable hCD80ECD:hIgG1Fc serum concentration available. Individual and group mean hCD80ECD:hIgG1Fc serum concentration versus time data following first dose (Cycle 1) for these patients are presented in FIG. 13.

Pharmacokinetic analysis indicated that hCD80ECD:hIgG1Fc has linear clearance with an estimated terminal half-life of approximately 1 week for doses with adequate pharmacokinetic data (7 mg-560 mg with n≥3). Results are consistent with minimal to no accumulation following repeated Q3W (one dose every three weeks) dosing.

Additionally, there were no dose limiting toxicities identified through the 560 mg dose level and no clinical evidence of cytokine release syndrome. Regarding adverse events, 14 out of 46 patients treated with hCD80ECD:hIgG1Fc (30%) had a serious adverse event (SAE) including 3/10 (30%) patients at 280 mg and 2/11 (18%) patients at 560 mg. Among these SAEs, two were attributed hCD80ECD:hIgG1Fc at 560 mg. One patient reported a SAE of grade 3 hepatitis following rechallenge with hCD80ECD:hIgG1Fc. This patient initially developed a Grade 1 rash at the end of cycle 1, which then worsened to Grade 3 in the week following the second dose. The patient was treated with oral corticosteroids with prompt improvement and was rechallenged with 560 mg of hCD80ECD:hIgG1Fc and developed a grade 3 elevation in alanine transaminase (ALT) and grade 2 elevation in aspartate transaminase (AST) without hyperbilirubinemia. The patient remained asymptomatic throughout with the liver function test elevations identified in protocol-specified laboratory testing one week following the third dose. This patient was hospitalized for management of the presumed autoimmune hepatitis with improvement following administration of IV corticosteroids and was discharged from the hospital to complete a taper of oral corticosteroids. hCD80ECD:hIgG1Fc was discontinued at the time of development of hepatitis.

A second patient reported a Grade 3 SAE of anaphylactic reaction following the fourth dose of hCD80ECD:hIgG1Fc at 560 mg with dyspnea, chills, wheezing and tachycardia. The patient was treated for an acute infusion reaction with corticosteroids, an antihistamine and inhaled b2-agonist, had resolution of their symptoms, and was discharged home from the emergency room following observation.

Grade ≥3 immune adverse events at 560 mg of hCD80ECD:hIgG1Fc other than the rash, hepatitis and anaphylactic reaction noted above include one additional patient with a Grade 3 infusion reaction (reported as infusion site reaction) and one additional patient with a Grade 3 rash. Both patients were treated with corticosteroids, had prompt resolution of their symptoms, did not require hospital admission for management of their immune-related adverse events, and subsequently resumed treatment with hCD80ECD:hIgG1Fc at 560 mg. No other grade ≥3 adverse events attributed to hCD80ECD:hIgG1Fc have been reported at 560 mg.

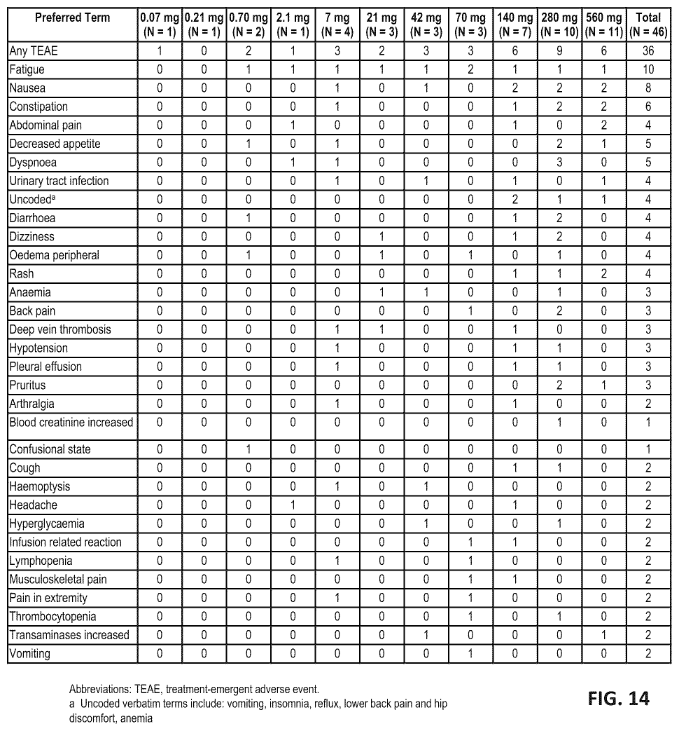

Adverse events irrespective of attribution reported in more than one patient are presented in FIG. 14.

Adverse events attributed to administration with hCD80ECD:hIgG1Fc in more than 1 patient include rash (n=5; reported as rash, erythematous, generalized, macular), fatigue (5), nausea (5), decreased appetite (3), pruritus (3), rash (3), diarrhea (2), and infusion related reaction (2). Both cases of diarrhea improved with supportive care and without the use of corticosteroids-a grade 2 event reported in a patient treated with hCD80ECD:hIgG1Fc at 0.7 mg and a grade 1 event reported in a patient treated with hCD80ECD:hIgG1Fc at 140 mg.

Immune-related adverse events (irAE) attributed to hCD80ECD:hIgG1Fc treatment have included rash, infusion related reactions and following rechallenge after interruption due to grade 3 rash, 1 case of hepatitis. Rash has been observed at the 280 mg and 560 mg dose levels with onset in the first 4 weeks following initiation of treatment with two cases of grade 3 rash, one case of grade 2 rash and two cases of grade 1 rash. Grade 2 and 3 events have responded to administration of systemic corticosteroids per consensus guidelines for irAEs. Infusion-related reactions including hypersensitivity and anaphylactic reactions have occurred in four patients with one grade 1 event at 70 mg, one grade 2 event at 140 mg, and two grade 3 events at 560 mg. The grade 1 and grade 2 events occurred at the time or subsequent to the second dose; the grade 3 events occurred at the time or following the fourth dose. All patients improved following administration of corticosteroids, antihistamines and supportive care as appropriate per institutional standard of care for infusion reactions. These results indicate that hCD80ECD:hIgG1Fc has a surprisingly tolerable overall safety profile. Therefore, hCD80ECD:hIgG1Fc can be a therapeutic for stimulating and improving a patient's own anti-tumor immune response. And hCD80ECD:hIgG1Fc is tolerable to patients with extended treatment resulting in tumor size reduction and patient benefit.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes.

Other embodiments are within the following claims.

TABLE OF SEQUENCES
The table below provides a listing
of certain sequences referenced herein.

| SEQ. ID. NO. | Description | Sequence |
|---|---|---|
| 1 | Human CD80 | VIHVTKEVKEVATLSCGHNVSVEELAQTRIYWQKEKKMVLTMM |
| | ECD sequence | SGDMNIWPEYKNRTIFDITNNLSIVILALRPSDEGTYECVVLK |
| | (without signal | YEKDAFKREHLAEVTLSVKADFPTPSISDFEIPTSNIRRIICS |
| | sequence) | TSGGFPEPHLSWLENGEELNAINTTVSQDPETELYAVSSKLDF |
| | | NMTTNHSFMCLIKYGHLRVNQTFNWNTTKQEHFPDN |
| 2 | Mouse CD80 | VDEQLSKSVKDKVLLPCRYNSPHEDESEDRIYWQKHDKVVLSV |
| | ECD sequence | IAGKLKVWPEYKNRTLYDNTTYSLIILGLVLSDRGTYSCVVQK |
| | (without signal | KERGTYEVKHLALVKLSIKADFSTPNITESGNPSADTKRITCF |
| | sequence) | ASGGFPKPRFSWLENGRELPGINTTISQDPESELYTISSQLDF |
| | | NTTRNHTIKCLIKYGDAHVSEDFTWEKPPEDPPDSKN |
| 3 | Fc human | EPKSSDKTHTCPPCPAPELLGGPSVFLFPPKPKDTLMISRTPE |
| | IgG1 | VTCVVVDVSHEDPEVKFNWYVDGVEVHNAKTKPREEQYNSTYR |
| | | VVSVLTVLHQDWLNGKEYKCKVSNKALPAPIEKTISKAKGQPR |
| | | EPQVYTLPPSRDELTKNQVSLTCLVKGFYPSDIAVEWESNGQP |
| | | ENNYKTTPPVLDSDGSFFLYSKLTVDKSRWQQGNVFSCSVMHE |
| | | ALHNHYTQKSLSLSPGK |
| 4 | Mouse CD80 | VDEQLSKSVKDKVLLPCRYNSPHEDESEDRIYWQKHDKVVLSV |
| | ECD mouse Fc | IAGKLKVWPEYKNRTLYDNTTYSLIILGLVLSDRGTYSCVVQK |
| | IgG2a (Fc | KERGTYEVKHLALVKLSIKADFSTPNITESGNPSADTKRITCF |
| | portion | ASGGFPKPRFSWLENGRELPGINTTISQDPESELYTISSQLDF |
| | underlined) | NTTRNHTIKCLIKYGDAHVSEDFTWEKPPEDPPDSKNEPRGPT |
| | | IKPCPPCKCPAPNLLGGPSVFIFPPKIKDVLMISLSPIVTCVV |
| | | VDVSEDDPDVQISWFVNNVEVHTAQTQTHREDYNSTLRVVSAL |
| | | PIQHQDWMSGKEFKCKVNNKDLPAPIERTISKPKGSVRAPQVY |
| | | VLPPPEEEMTKKQVTLTCMVTDFMPEDIYVEWTNNGKTELNYK |
| | | NTEPVLDSDGSYFMYSKLRVEKKNWVERNSYSCSVVHEGLHNH |
| | | HTTKSFSRTPGK |
| 5 | Human CD80 | VIHVTKEVKEVATLSCGHNVSVEELAQTRIYWQKEKKMVLTMM |
| | ECD Human | SGDMNIWPEYKNRTIFDITNNLSIVILALRPSDEGTYECVVLK |
| | Fc IgG1 WT | YEKDAFKREHLAEVTLSVKADFPTPSISDFEIPTSNIRRIICS |
| | (Fc portion | TSGGFPEPHLSWLENGEELNAINTTVSQDPETELYAVSSKLDF |
| | underlined) | NMTTNHSFMCLIKYGHLRVNQTFNWNTTKQEHFPDNEPKSSDK |
| | | THTCPPCPAPELLGGPSVFLFPPKPKDTLMISRTPEVTCVVVD |
| | | VSHEDPEVKFNWYVDGVEVHNAKTKPREEQYNSTYRVVSVLTV |
| | | LHQDWLNGKEYKCKVSNKALPAPIEKTISKAKGQPREPQVYTL |
| | | PPSRDELTKNQVSLTCLVKGFYPSDIAVEWESNGQPENNYKTT |
| | | PPVLDSDGSFFLYSKLTVDKSRWQQGNVFSCSVMHEALHNHYT |
| | | QKSLSLSPGK |
| 6 | human PD-1 | PGWFLDSPDR PWNPPTFSPA LLVVTEGDNA |
| | (mature, | TFTCSFSNTS ESFVLNWYRM SPSNQTDKLA |
| | without signal | AFPEDRSQPG QDCRFRVTQL PNGRDFHMSV |
| | sequence) | VRARRNDSGT YLCGAISLAP KAQIKESLRA |
| | | ELRVTERRAE VPTAHPSPSP RPAGQFQTLV |
| | | VGVVGGLLGS LVLLVWVLAV ICSRAARGTI |
| | | GARRTGQPLK EDPSAVPVFS VDYGELDFQW |
| | | REKTPEPPVP CVPEQTEYAT IVFPSGMGTS |
| | | SPARRGSADG PRSAQPLRPE DGHCSWPL |
| 7 | human PD-L1 | FT VTVPKDLYVV EYGSNMTIEC KFPVEKQLDL |
| | (mature, | AALIVYWEME DKNIIQFVHG EEDLKVQHSS |
| | without signal | YRQRARLLKD QLSLGNAALQ ITDVKLQDAG |
| | sequence) | VYRCMISYGG ADYKRITVKV NAPYNKINQR |
| | | ILVVDPVTSE HELTCQAEGY PKAEVIWTSS |
| | | DHQVLSGKTT TTNSKREEKL FNVTSTLRIN |
| | | TTTNEIFYCT FRRLDPEENH TAELVIPELP |
| | | LAHPPNERTH LVILGAILLC LGVALTFIFR |
| | | LRKGRMMDVK KCGIQDTNSK KQSDTHLEET |

1. A method of treating a solid tumor in a human patient, the method comprising administering to the patient about 140 mg to about 1,260 mg of a fusion protein comprising the extracellular domain (ECD) of human cluster of differentiation 80 (CD80) and the fragment crystallizable (Fc) domain of human immunoglobulin G1 (IgG1).

2.-12. (canceled)

13. The method of claim 1, wherein about 840 mg to about 1,260 mg of the fusion protein is administered.

14. The method of claim 1, wherein about 700 mg to about 840 mg of the fusion protein is administered.

15. The method of claim 1, wherein about 630 mg to about 700 mg of the fusion protein is administered.

16. The method of claim 1, wherein about 560 mg to about 630 mg of the fusion protein is administered.

17. The method of claim 1, wherein about 420 mg to about 560 mg of the fusion protein is administered

18. The method of claim 1, wherein about 280 mg to about 420 mg of the fusion protein is administered.

19. The method of claim 1, wherein about 210 mg to about 280 mg of the fusion protein is administered

20. The method of claim 1, wherein about 140 mg to about 210 mg of the fusion protein is administered

21. The method of claim 1, wherein the fusion protein is administered once every three weeks.

22. The method of claim 1, wherein the fusion protein is administered intravenously.

23. The method of claim 1, wherein the ECD of human CD80 comprises the amino acid sequence set forth in SEQ ID NO:1.

24. The method of claim 1, wherein the Fc domain of human IgG1 comprises the amino acid sequence set forth in SEQ ID NO:3.

25. The method of claim 1, wherein the Fc domain of human IgG1 is linked to the carboxy terminus of the ECD of human CD80.

26. The method of claim 1, wherein the fusion protein comprises the amino acid sequence set forth in SEQ ID NO:5.

27. The method of claim 1, wherein the fusion protein comprises at least 20 molecules of SA.

28. The method of claim 1, wherein the fusion protein comprises at least 15 molecules of SA.

29.-32. (canceled)

33. The method of claim 1, wherein the fusion protein is administered in a pharmaceutical composition that further comprises a pharmaceutically acceptable excipient.

34. The method of claim 33, wherein the pharmaceutical composition comprises at least 20 moles of SA per mole of fusion protein.

35. The method of claim 33, wherein the pharmaceutical composition comprises at least 15 moles of SA per mole of fusion protein.

36.-39. (canceled)

40. The method of claim 1, wherein the solid tumor is an advanced solid tumor.

41. The method of claim 1, wherein the solid tumor is not a primary central nervous system tumor.

42. The method of claim 1, wherein the solid tumor is a colorectal cancer, breast cancer, gastric cancer, non-small cell lung cancer, small cell lung cancer, melanoma, squamous cell carcinoma of the head and neck, ovarian cancer, pancreatic cancer, renal cell carcinoma, hepatocellular carcinoma, bladder cancer, endometrial cancer, or sarcoma.

43. The method of claim 1, wherein the solid tumor is a renal cell carcinoma.

44. The method of claim 1, wherein the solid tumor is melanoma.

45. The method of claim 1, wherein the patient has not received prior therapy with a PD-1/PD-L1 antagonist.

46. The method of claim 1, wherein the patient has received prior therapy with at least one PD-1/PD-L1 antagonist selected from a PD-L1 antagonist and a PD-1 antagonist.

47. The method of claim 46, wherein the at least one PD-1/PD-L1 antagonist is nivolumab, pembrolizumab, atezolizumab, durvalumab, or avelumab.

48. The method of claim 46, wherein the at least one PD-1/PD-L1 antagonist was administered in an advanced or metastatic setting.

49. The method of claim 1, wherein the patient has received prior therapy with at least one anti-angiogenic agent.

50. The method of claim 49, wherein the anti-angiogenic agent is sunitinib, sorafenib, pazopanib, axitinib, tivozanib, ramucirumab, or bevacizumab.

51. The method of claim 49, wherein the anti-angiogenic agent was administered in an advanced or metastatic setting.

52. The method of claim 44, wherein the patient has a BRAF mutation.

53. The method of claim 52, wherein the patient has received prior therapy with at least one BRAF inhibitor.

54. The method of claim 53, wherein the BRAF inhibitor is vemurafenib or dabrafenib.

55. The method of claim 53, wherein the BRAF inhibitor was administered in an advanced or metastatic setting.

56. The method of claim 1, wherein the solid tumor is recurrent or progressive after a therapy selected from surgery, chemotherapy, radiation therapy, and a combination thereof.


SEQUENCE LISTING (7 sequences)

SEQ ID NO: 1 - protein, 208 aa, Artificial Sequence [sequence omitted]

SEQ ID NO: 2 - protein, 209 aa, Artificial Sequence
  Mouse CD80 ECD sequence (without signal sequence)
VDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKN

SEQ ID NO: 3 - protein, 232 aa, Artificial Sequence
  Fc human IgG1
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 442 aa, Artificial Sequence
  Mouse CD80 ECD mouse Fc IgG2a
VDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 5 - protein, 440 aa, Artificial Sequence
  Human CD80 ECD Human Fc IgG1 WT
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 268 aa, Artificial Sequence
  human PD-1 (mature, without signal sequence)
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 7 - protein, 272 aa, Artificial Sequence
  human PD-L1 (mature, without signal sequence)
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET